(19) United States
(12) Patent Application Publication
(10) Pub. No.: US 2025/0288325 A1
(43) Pub. Date: Sep. 18, 2025

(54) SYSTEM AND METHOD FOR THE PREVENTION OR MANAGEMENT OF UTERINE HEMORRHAGE

(71) Applicant: Boehringer Technologies, LP, Phoenixville, PA (US)

(72) Inventors: Christopher L. RADL, Malvern, PA (US); Trevor Smith, West Chester, PA (US); Jaime L. Schlorff, Phoenixville, PA (US)

(21) Appl. No.: 19/220,270

(22) Filed: May 28, 2025

Related U.S. Application Data
(63) Continuation-in-part of application No. 18/212,390, filed on Jun. 21, 2023.
(60) Provisional application No. 63/354,774, filed on Jun. 23, 2022.

(51) Int. Cl. A61B 17/42; A61B 1/06; A61B 17/00; A61B 17/12; A61M 1/00

(52) U.S. Cl. CPC A61B 17/4241; A61B 1/0684; A61B 17/1204; A61B 17/12131; A61M 1/743; A61M 1/75; A61B 2017/00566; A61B 2017/12004; A61B 2017/4216

(57) ABSTRACT

A device and method of use for preventing or treating postpartum uterine bleeding or hemorrhage of a patient. The device includes a suction tube having a distal end portion at which an aperture or an array of plural apertures is located and extending about the periphery of the distal portion. A sealing member having an apex and flexible sidewall is mounted on the suction tube so that a portion of its sidewall can be brought into engagement with tissue of the uterus. Suction from a vacuum source is applied to a proximal end portion of the suction tube to apply suction via the aperture(s) to the interior of the patient's uterus. The suction tube may include an illumination system for illuminating the interior of the patient's vagina.

### CROSS-REFERENCE TO RELATED APPLICATIONS

This application is a Continuation-In-Part of U.S. patent application Ser. No. 18/212,390 filed on Jun. 21, 2023, entitled System And Method For Management Of Uterine Hemorrhage, which in turn claims the benefit under 35 U.S.C. § 119(e) of Provisional Application Ser. No. 63/354,774, filed on Jun. 23, 2022, entitled “System And Method For Management Of Uterine Hemorrhage, all of which applications are assigned to the same assignee as the subject invention and whose entire disclosures are specifically incorporated by reference herein for all purposes.

### FIELD OF THE INVENTION

This invention relates generally to medical devices and methods and more particularly to devices and methods for preventing or managing excessive uterine bleeding.

### SPECIFICATION

### BACKGROUND OF THE INVENTION

Postpartum hemorrhage (PPH) is one of the leading causes of maternal mortality and morbidity worldwide and manifests itself as uterine blood loss exceeding normal levels, typically 500 ml following vaginal delivery or 1000 ml following cesarean delivery. The most common cause of PPH is a condition referred to as uterine atony in which the uterus fails to contract sufficiently to stop bleeding from the vessels at the placenta implantation site. Different interventions have been used to treat PPH with varying levels of success. These include administration of uterotonic medications to stimulate contraction, use of hemostatic agents to arrest bleeding, external uterine massage and bimanual compression, and intrauterine balloon tamponade devices. If these first line measures are unsuccessful, surgical techniques such as uterine and pelvic artery ligation and uterine compression sutures may be used. In severe cases, blood transfusions and emergency hysterectomy may be required.

U.S. Pat. No. 9,125,686 (Norred et al.), U.S. Pat. No. 9,550,014 (Norred et al.), U.S. Pat. No. 10,064,651 (Norred et al.); U.S. Pat. No. 11,241,254 (Norred et al.); and U.S. Pat. No. 11,291,473 (Norred et al.), which are assigned to Alydia Health, Inc. disclose systems and methods for treating PPH by creating negative pressure within the uterus to induce uterine contraction to constrict the vasculature of the uterus and control bleeding. For example, those patents disclose a uterine hemorrhage controlling system comprising an intrauterine suction module connected to a cervical sealing module that is couplable to a vacuum source. The suction module includes a tube, either straight or configured as a loop, e.g., an intrauterine loop, with a set of holes to deliver suction into the uterus when the sealing module is connected to a vacuum source. The sealing module includes an expandable balloon located outside of the uterus to hermetically seal the vaginal entrance to the uterus. A uterine hemorrhage controlling method comprises inserting a suction module into the uterus with a sealing module to occlude the entrance into the uterus, connecting the suction module to a vacuum source and initiating suction to create negative pressure within the uterus.

Alydia Health, Inc. provides a vacuum-induced hemorrhage control system under the designation The Jada® System which is constructed in accordance with the teachings of the aforementioned Norred et al. patents. The published Instructions for Use of the Jada® system describe it as follows: “The Jada System is a 41 cm long intrauterine device made of silicone. Jada consists of an Intrauterine Loop on the distal end of a Tube. The proximal end of the Tube has a Vacuum Connector for connection to sterile vacuum tubing. The Cervical Seal proximal to the Intrauterine Loop is filled and emptied with a sterile luer tapered syringe filled with sterile fluid via the Seal Valve. The Intrauterine Loop consists of a loop tube with 20 Vacuum Pores oriented toward the inside diameter of the Intrauterine Loop. The outer surface of the Intrauterine Loop is covered by a Shield which overhangs the Vacuum Pores to protect tissue from vacuum and the Vacuum Pores from plugging with tissue and blood clots. A sterile luer tapered 60 mL syringe and a sterile 12′ vacuum tubing are supplied with the Jada System.” Among the Instructions for Use under the heading “Jada Placement: Post Vaginal Delivery or Post Cesarean Section After Closure of Hysterotomy” are the following instructions: “ . . . 7. Secure visualization of the cervix to confirm it is dilated ≥3 cm to allow for placement of Jada. 8. Using a hand, compress the Intrauterine Loop near the distal tip for support and insert Jada transvaginally, leading with the Intrauterine Loop. Avoid excessive force. Use gentle traction on the anterior cervical lip to stabilize the cervical opening, if needed. An instrument can be placed on the anterior cervical lip, but do not grasp Jada with an instrument to facilitate intrauterine insertion. 9. Place Jada such that the Intrauterine Loop is located in the uterus and is oriented in the frontal plane of the body by assuring the Seal Valve is oriented at either 3 or 9 o'clock. Ultrasound may be used to confirm proper placement of the Intrauterine Loop within the uterus. 10. After insertion, the Intrauterine Loop should be within the uterus while the Cervical Seal should be located within the vagina at the external cervical os.”

While the Jada® Systems and methods of use disclosed in the aforementioned Norred et al. patents appear generally suitable for their intended purpose they nevertheless leave something to be desired from the standpoints of simplicity of construction and ease of use.

Accordingly, a need exists for device and method of use for the management of PPH which is effective in use, simple in construction, low in cost, easy to use, and which overcomes deficiencies of the prior art. The subject invention addresses that need.

### BRIEF SUMMARY OF THE INVENTION

One aspect of this invention is a device for preventing or treating uterine bleeding or hemorrhage of a female patient having a uterus. The device comprises a suction tube or conduit, at least one light emitting component, and a sealing member. The suction tube or conduit comprises an elongated sidewall having a periphery, a distal free end portion, a proximal end portion, a passageway extending about a longitudinal axis within the sidewall, and at least one aperture in fluid communication with the passageway located at either the distal free end portion of the sidewall or at another portion of the sidewall closely adjacent the distal free end portion, or at the distal free end portion of the sidewall and the another portion of said sidewall. The at least one light emitting component is coupled to the suction tube and configured to be illuminated, whereupon light from the light emitting component provides light outside of the suction tube. The sealing member has a sidewall, an apex at a top of the sidewall, and an exterior surface located proximally of the apex. The distal free end portion of the suction tube extends outward distally of the apex. The device is configured to enable the distal free end portion of the suction tube to be positioned into the uterus of the female patient such that the at least one aperture is confined to the interior of the uterus. The sealing member is configured to be positioned with respect to the uterus so that tissue of the female patient conforms around and engages the exterior surface of the sealing member. The light emitting component illuminates tissue of the female patient. The proximal end of the suction tube is configured so that suction can be applied thereto from a suction source and through the passageway to the at least one aperture to apply suction through the at least one aperture into the uterus of the female patient, whereupon the engagement of the exterior surface of the sealing member with tissue of the female patient forms a seal therebetween to maintain suction within the uterus.

In accordance with one preferred aspect of the device of this invention the light emitting component is located within the sidewall, and the sidewall is configured to enable light to pass out of said sidewall.

In accordance with another preferred aspect of the device of this invention the at least one aperture comprises an array of apertures extending through the distal free end portion of the sidewall about the entire periphery of the sidewall along a predetermined length of the suction tube at the distal free end portion.

In accordance with another preferred aspect of the device of this invention the sealing member is slidably mounted on the suction tube.

In accordance with another preferred aspect of the device of this invention the predetermined length of the array of apertures is in the range of approximately 4 cm to 14 cm.

In accordance with another preferred aspect of the device of this invention the sidewall of the sealing member is flexible.

In accordance with another preferred aspect of the device of this invention the at least one light emitting component comprises a series of light emitting diodes.

In accordance with another preferred aspect of the device of this invention the series of light emitting diodes is located within the central passageway of the suction tube, and wherein at least a portion of the sidewall of the suction tube is transparent or translucent to enable light from the light emitting diodes to pass therethrough.

In accordance with another preferred aspect of the device of this invention the device additionally comprises a source of electrical power to provide electricity to the series of light emitting diodes. The source of electrical power is located adjacent the proximal end portion.

In accordance with another preferred aspect of the device of this invention the device additionally comprises a suction regulator coupled to distal end portion of the suction tube to regulate suction provided by the suction source and to provide regulated suction through the passageway to the at least one aperture.

In accordance with another preferred aspect of the device of this invention the suction source comprises a manually operable pump configured to be coupled to the proximal end of the suction tube to apply suction to the suction tube.

In accordance with another preferred aspect of the device of this invention the manually operable pump comprises a hollow squeezable body including an interior chamber having an inlet port and an outlet port, with the inlet port being coupled to the proximal end of the suction tube via one-way inlet valve, and with the outlet port being coupled to the ambient atmosphere via a one-way outlet valve.

In accordance with another preferred aspect of the device of this invention the device additionally comprises a shield located adjacent the apex of the suction tube to deter the blockage of the array of apertures by debris.

In accordance with another preferred aspect of the device of this invention the shield comprises a dome shaped member having a peripheral skirt portion overlying the array of apertures.

Another aspect of this invention is method for preventing or treating uterine bleeding or hemorrhage in a female patient. The female patient has a uterus having an interior and an opening. The method comprises providing a suction tube comprising an elongated sidewall having a periphery, a distal free end portion, a proximal end portion, a passageway extending about a central longitudinal axis within the sidewall, at least one aperture in fluid communication with the passageway located at either the distal free end portion of the sidewall or a portion of the sidewall closely adjacent the distal free end portion. The suction tube includes at least one light emitting component coupled to the suction tube and configured to be illuminated, whereupon light from the light emitting component provides light outside of the suction tube. A sealing member having a sidewall, an apex at a top of the sidewall and an exterior surface located proximately of the apex is provided on the suction tube. The distal end portion of the suction tube is inserted into the uterus of the female patient such that the at least one aperture is confined to the interior of the uterus. The sealing member is positioned with respect to the uterus so that tissue of the female patient at the opening of the uterus conforms around and engages the exterior surface of the sealing member. The light emitting component is caused to illuminate the tissue outside the uterus. Suction is applied to the proximal end of the suction tube to apply suction through the at least one aperture into the uterus of the female patient, whereupon the engagement of the exterior surface of the sealing member with the tissue of the female patient forms a seal therebetween to maintain suction within the uterus.

In accordance with one preferred aspect of the method of this invention the light emitting component is located within the sidewall, and the sidewall is configured to enable light to pass out of said sidewall.

In accordance with another preferred aspect of the method of this invention the method additionally comprising providing a receptacle or canister for collection of fluid or other body materials resulting from the application of suction to the uterus of the patient.

In accordance with another preferred aspect of the method of this invention, the suction is applied to the uterus continuously or intermittently.

In accordance with another preferred aspect of the method of this invention, the suction is applied to the uterus by a manually operable pump.

### BRIEF DESCRIPTION OF SEVERAL VIEWS OF THE DRAWINGS

FIG. 1A is an illustration of the reproductive system of a female human patient making use of one exemplary PPH device of this invention in a system for managing the patient's postpartum bleeding or hemorrhage, and showing one method of use of the PPH device whereupon a sealing member forming a part of the PPH device is located outside the body of the patient, but with a portion of it in engagement with the introitus;

FIG. 1B is an illustration similar to FIG. 1A, but showing another method of use of the device of this invention but with its sealing member being located partially within the patient's vagina;

FIG. 1C is another illustration, similar to FIGS. 1A and 1B, but showing another method of use of the device of this invention but with its sealing member being located completely within the patient's vagina;

FIG. 2 is an enlarged isometric view of the PPH device shown in FIG. 1A;

FIG. 3 is a more enlarged isometric view, in longitudinal section, of a portion of the PPH shown in FIG. 2, namely an intrauterine tip, a vacuum tube and the aforementioned sealing member;

FIG. 4 is an enlarged isometric view of an optional portion of the system shown in FIG. 1A, namely, a suction regulator, an on-off selector valve, and a tubes or conduits for coupling the PPH device of FIG. 2 to a source of suction and to a receptacle or canister, like shown in FIG. 1A;

FIG. 5 is an enlarged longitudinal section view of the sealing member shown in FIG. 3;

FIG. 6 is an enlarged longitudinal section view of the intrauterine tip shown in FIG. 3;

FIG. 7 is a greatly enlarged transverse sectional view of a suction tube forming a portion of the PPH device and taken along line 7-7 of FIG. 2;

FIG. 8 is an enlarged longitudinal sectional view of the optional suction regulator shown in FIG. 4;

FIG. 9 is an isometric view, similar to FIG. 2, but showing an alternative PPH device making use of an alternative sealing member;

FIG. 10 is an enlarged side elevation view of the alternative sealing member shown in FIG. 9;

FIG. 11 is a sectional view taken along line 11-11 of FIG. 10;

FIG. 12 is a vertical sectional view similar to FIG. 11, but showing dimensions of one exemplary embodiment of the alternative sealing member of FIGS. 9 and 10;

FIG. 13 is an isometric view, similar to FIG. 2, but showing still another alternative PPH device making use of another alternative sealing member;

FIG. 14 is an enlarged side elevation view of the alternative sealing member shown in FIG. 13;

FIG. 15 is an enlarged sectional view taken along line 15-15 of FIG. 14;

FIG. 16 is a side elevation view of the alternative sealing member of FIGS. 14 and 15 showing the dimensions thereof;

FIG. 17 is an isometric view, similar to FIGS. 2, but showing another alternative PPH device making use of another preferred alternative sealing member, with another preferred optional suction regulator, but without the on-off selector valve and a tubes or conduits for coupling the PPH device to a source of suction and to a receptacle or canister being shown, like they are shown in FIG. 1A;

FIG. 18 is a somewhat enlarged longitudinal sectional view of the PPH device of FIG. 17, taken along line 18-18 of FIG. 17;

FIG. 19 is an enlarged exploded isometric view of the components of the PPH device of FIG. 17 without the optional suction regulator;

FIG. 20 is an enlarged top plan view of the optional suction regulator of the PPH device of FIG. 17, which suction regulator is shown in section within the broken circle designated by the reference number 20 in FIG. 18;

FIG. 21 is an exploded isometric view of the suction regulator of FIG. 20;

FIG. 22 is an enlarged sectional view of the suction regulator of FIGS. 17 and 20 taken along the longitudinal axis X in FIG. 20;

FIG. 23 is a longitudinal sectional view, similar to FIG. 12, of the alternate sealing member of FIGS. 17 and 20;

FIG. 24 is an illustration of the reproductive system of a female human patient making use of another exemplary PPH device of this invention in a system for managing the patient's postpartum bleeding or hemorrhage, wherein the system includes among other things a suction tube having an internally located illumination source, an encasement sleeve holding the illumination source, an electrical subsystem for powering the illumination source, and a sealing member, with the sealing member being located on the suction tube and completely within the patient's vagina;

FIG. 25 is an enlarged side elevational view of a portion of the PPH device shown in FIG. 24, namely an illuminated suction tube and an associated sealing member;

FIG. 26 is an enlarged isometric sectional view of the sealing member of the PPH device shown in FIG. 24;

FIG. 27 is an enlarged isometric view, partially in section, of the portion of the PPH device shown in FIG. 25 and in particular the sealing member, the suction tube, the internally located illumination source and its encasement sleeve;

FIG. 28 is an isometric view of a portion of the PPH device shown in FIG. 24, but without the sealing member of the PPH device;

FIG. 29 is an isometric view portion of the PPH device shown in FIG. 24, with the electrical subsystem shown exploded, but without the suction tube, the sealing member and the encapsulation sleeve being shown;

FIG. 30 is an enlarged isometric view of a portion of the illumination source shown within the broken line oval designated by the reference number 30;

FIG. 31 is a schematic diagram of the electrical components making up the electrical subsystem including an illumination source;

FIG. 32 is an enlarged isometric view of the encasement sleeve;

FIG. 33 is an enlarged isometric view of the illumination source, which is in the form of a flexible circuit including plural light emitting diodes;

FIG. 34 is an isometric view of a portion of the illumination source, namely, the assembly of a flexible circuit and its associated electrically conductive cable, with the flexible circuit being shown located within the encasement sleeve;

FIG. 35 is a greatly enlarged plan view of the distal end of the assembly shown in FIG. 34;

FIG. 36 is a greatly enlarged side elevation view of the distal end of the assembly shown in FIG. 34;

FIG. 37 is an illustration showing the light emitted from the light emitting diodes of the flexible circuit shown in FIGS. 33-36;

FIG. 38 is an enlarged exploded isometric view of a portion of the components making up the electrical power source of the electrical subsystem and the housing thereof;

FIG. 39 is an enlarged end view taken along line 39-39 of FIG. 28;

FIG. 40 is an illustration similar to FIG. 24 but showing an alternative PPH device making use of an alternative embodiment of the suction tube, with the sealing member being located on the suction tube and completely within the patient's vagina;

FIG. 41 is an isometric view of the suction tube of the PPH device of FIG. 40;

FIG. 42 is an illustration similar to FIG. 40, but showing another alternative PPH device wherein that PPH device makes use of the same components of the device of FIG. 40 but with an alternative means for providing suction to the suction tube, namely, a manually squeezable bulb in place of the suction regulator and associated components, and with other components of FIG. 40, e.g., the receptacle and associated tubing sections omitted in the interest of drawing simplicity;

FIG. 43 is an enlarged longitudinal sectional view of the manually squeezable bulb taken along line 43-43 of FIG. 42; and

FIG. 44 is an isometric longitudinal sectional view of an alternative to the suction tube of FIG. 41 whose distal end includes a shield to prevent the clogging of the apertures and distal opening in the nub portion of the suction tube.

### DETAILED DESCRIPTION OF THE PREFERRED EMBODIMENTS OF THE INVENTION

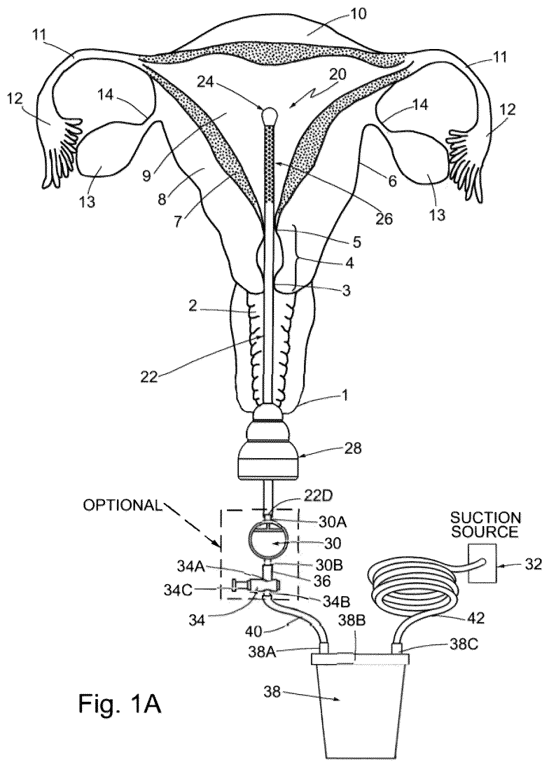

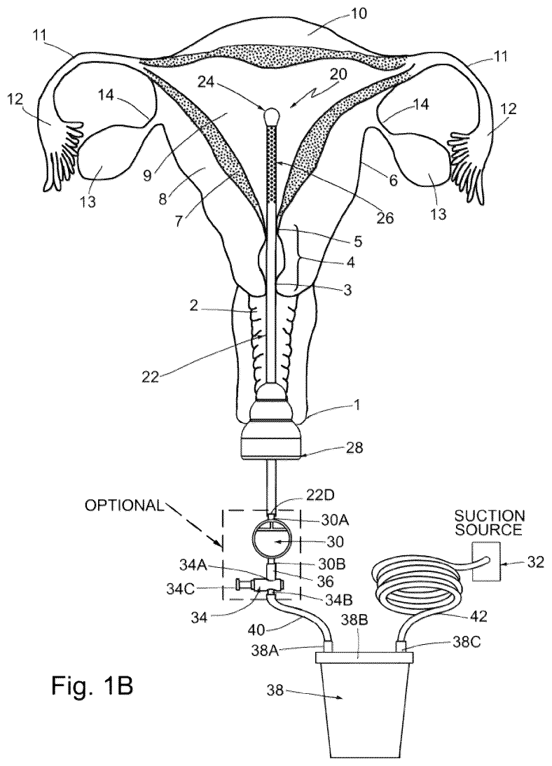

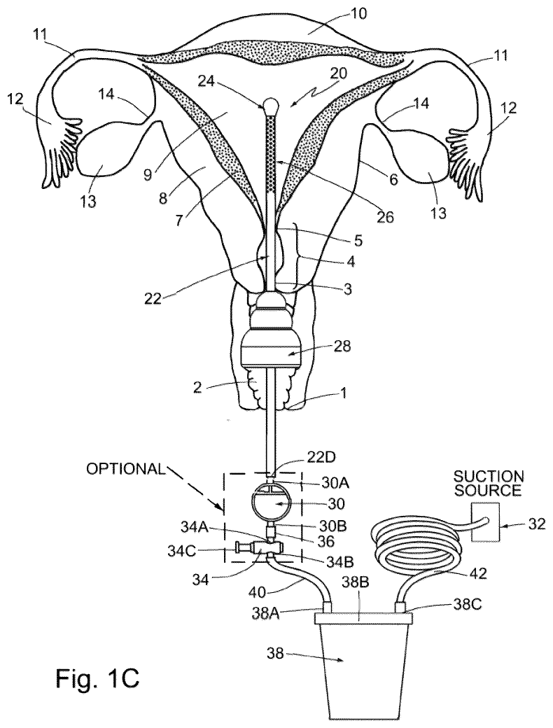

Referring now to the various figures of the drawing wherein like reference characters refer to like parts, there is shown in FIG. 1 one exemplary embodiment of a PPH device 20 constructed in accordance with this invention for managing postpartum bleeding or hemorrhage in a female human being patient, with the device being shown in place in the patient's reproductive system. It should be pointed out at this juncture, that while the subject invention has particular application for the management of PPH, it is not so limited. Thus, the PPH devices disclosed and described hereinafter can be used to manage or treat any type of uterine or vaginal bleeding.

Before describing the PPH device 20 a brief summary of the female reproductive system as shown in FIG. 1A will now be described and its anatomical components identified by reference numbers in that figure. In particular, the introitus 1 is shown and forms the entrance to the vagina 2. A vaginal artery stems from the internal iliac artery and supplies blood to the vagina. The vagina 2 extends to the external os 3, which is the opening of the cervix 4 into the vagina 2. The internal os 5 is the opening of the cervix 4 into the uterus 9. The uterus 9 consists of a cavity that extends from the internal os 5 to the uterine fundus 10. The endometrium 7 constitutes the inner lining of the uterus 9, the myometrium 8 is the muscle layer of the uterus 9 surrounding the endometrium 7, and the perimetrium 6 is the outermost layer of the uterus 9 surrounding the myometrium 8. A spiral array of arteries surrounds the uterus 9 and supplies blood to the endometrium 7 which is used to provide oxygen and nutrients to the embryo and fetus during pregnancy. In FIG. 1A the area where the potentially bleeding arteries are located is shown by the stippling. Postpartum hemorrhage occurs in this region at the location at which the placenta was previously attached to the inside wall of the uterus. The ovaries 13 which produce eggs are located on either side of the uterus and connect to the uterus by ovarian ligaments 14. The fallopian tubes 11, which are the conduits for the egg to travel from the ovaries 13 to the uterus 9, extend on opposite sides of the uterus 9 which terminate in respective fimbriae 12 which are finger-like projections to catch the egg released during ovulation.

As will be described in considerable detail later, the PPH device 20 and its methods of use is designed to create negative pressure in the interior cavity of the uterus to induce uterine compression and contraction necessary to cause hemostasis of the spiral array of arteries previously attached to the placenta during pregnancy. This may be necessary when loss of tone in the uterine musculature, known as uterine atony, prevents adequate contraction and results in excessive bleeding or hemorrhage. Such action should reduce, if not halt or terminate, postpartum uterine hemorrhaging. To that end, a suction or vacuum tube forming a portion of the PPH device 20 is configured to be inserted into the uterus and connected to a source of suction so that when connected it forms a system for managing postpartum hemorrhage.

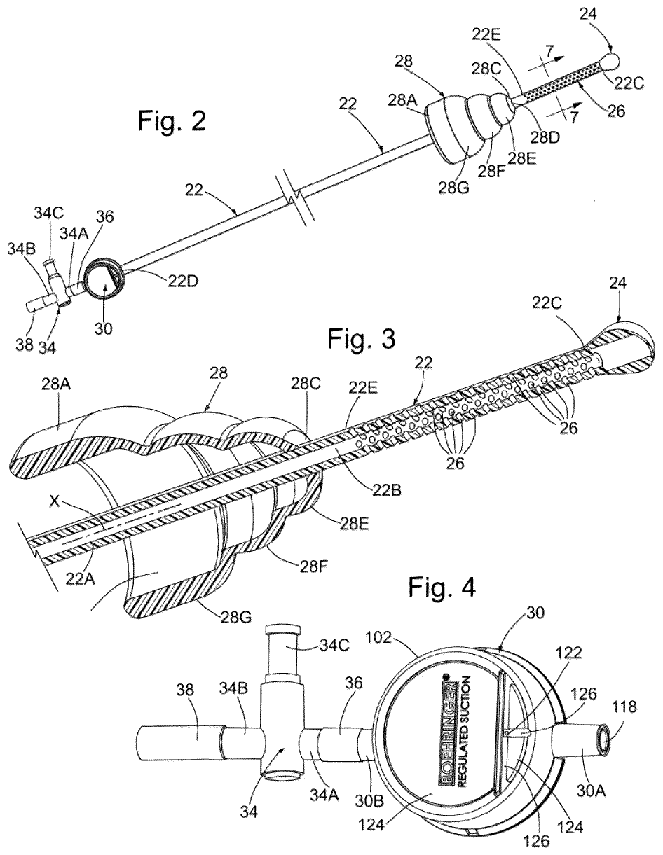

The details of the PPH device 20 of the subject invention will be described later. Suffice it for now to state that it can be configured in various ways but basically comprises a vacuum or suction tube 22 having a sidewall 22A which bounds a central longitudinally extending passageway 22B extending the length of the tube from its distal end 22C to its proximal end 22D. An intrauterine tip 24, which is atraumatic, is located at the distal end 22C of the suction tube and closes off the distal end of the central passageway 22B but does not necessarily close off the vacuum thereat. The suction tube includes at least one aperture, and preferably a plurality of apertures. For example, as best seen in FIGS. 2 and 3, the suction tube 22 includes a plurality of radially extending apertures or holes 26 located in an array (to be described later) extending through the sidewall 22A of the suction tube from its distal end 22C to some intermediate location 22E (FIGS. 2 and 3), e.g., approximately 4 to 14 cm proximally of the atraumatic tip 24. Each of the apertures or holes 26 is in fluid communication with the central passageway 22B of the suction tube. A sealing member 28 in the form of a sealing member is mounted on a distally located portion of the suction tube. The sealing member is preferably slidably mounted on the suction tube but can be fixedly located on the distal end portion of the suction tube, if desired. The sealing member basically comprises a hollow portion having a sidewall 28A. The sidewall is preferably flexible but need not be flexible. In either case in its normal, quiescent, state the sidewall 28A is of a predetermined shape which is preferably generally conically shaped and includes plural tiered arcuate sections (to be described later).

The proximal end of the suction tube 22 is configured to be connected to a vacuum source. That vacuum source may be provided in various ways. For example, the vacuum source may be a hospital's vacuum or suction line 32. If the line level of suction provided at hospital's suction line 32 is at a value which is suitable for application to the interior of the patient's uterus to achieve safe and effective uterine contraction to optimize treatment, e.g., a suction level of approximately 80 mmHg+/−10 mmHg, the proximal end 22D of the suction tube can be connected directly to the hospital's vacuum line 32. If the hospital's suction line 32 provides suction at a higher level, any suitable suction regulator can be interposed between the hospital's suction line and the proximal end 22D of the suction tube 22 to reduce or control the level of suction provided to the suction tube to the desired safe value. For example, in the exemplary embodiment shown in FIG. 1A, an optional suction regulator 30 is shown connected between the hospital's suction line 32 and the proximal end 22D of the suction tube 22 to reduce or control the line suction to a regulated safe level or value of 80 mmHg+/−10 mmHg. That controlled level of suction is applied through the central passageway 22B of the suction tube 22 and out through the aperture or holes 26. In accordance with one preferred aspect of this invention, the optional suction regulator 30 is constructed like that of the suction regulator of U.S. Pat. No. 9,999,533 (Radl et al.) assigned to the same assignee as this invention and whose entire disclosure is specifically incorporated herein for all purposes.

As also seen in FIG. 1A an optional on/off selector valve 34 is shown connected to the suction regulator. It serves to selectively couple the suction regulator 30 to the vacuum source 32 when suction is to be applied to the interior of the patient's uterus. The on/off selector valve 34 is a slide valve which is constructed similarly to the slide valve 24 of the aforementioned U.S. Pat. No. 9,999,533 (Radl et al.). To that end, the on/off selector valve 34 includes a first port 34A (FIG. 4), a second port 34B, and a manually movable slide 34C. The first port 34A is connected to a port 30B (to be described later) of the suction regulator 30 via a flexible tube section 36. The second port 34B of the valve 34 is configured to be coupled via another flexible tube section 40 to the vacuum source 32. The slide 34C is slidably interposed between the first and second ports 34A and 34B, respectively, and manually movable to control the state of the selector valve. In particular, the selector valve 34 is configured to be placed in either an open (on) state or a closed (off) state by a user manipulating the slide 34C to either an open position or a closed position. When the slide is moved (e.g., slid) to its open position the selector valve is in its open state, which enables the suction from the vacuum source 32 to be applied to the port 30B of suction regulator 30. That action is accomplished by the user of the PPH device when the PPH device is in place and ready to apply negative pressure at the regulated suction level to the uterus of the patient. When the movable member 34C is moved to its off position the selector valve is in its off state, whereupon it isolates the suction regulator 30 from vacuum source 32 when suction is no longer to be applied to the patient's uterus.

In accordance with one preferred aspect of this invention a receptacle or canister 38 is provided with the PPH device 20 to form the system shown in FIG. 1A to collect any blood or other body fluids withdrawn from the uterus by the suction applied thereto when the PPH device 20 is in operation. The receptacle or canister 38 is preferably provided as a part of the PPH system whether or not the optional suction regulator 30 and on/off selector valve 34 are used. For example, if the PPH system includes the PPH device 20 and the optional suction regulator 30 and on/off selector valve 34 like shown in FIG. 1A, the receptacle or canister 38 is connected as follows. The port 34B of the selector valve is connected via a section of tube 40 to a first port 38A of the receptacle or canister 38. The first port 38A is located in a lid 38B of the receptacle or canister 38. The lid closes off the top of the hollow body making up the receptacle or canister. The lid also includes a second port 38C which is connected via another length of flexible tubing 42 to the vacuum source 32. Thus, when the on/off selector valve is in the on or open state, suction from the vacuum source 32 is applied via the path including tube section 42, the port 38C, the interior of the receptacle or canister 38, the port 38A, the tube section 40, through the optional on/off selector valve 34 and the optional suction regulator 30 to the central passageway 22B of the suction tube 22, and out through the apertures 26 into the interior of the uterus 9. Any blood or other body fluid withdrawn from the uterus by the application of suction thereto will flow through that path back until it enters into the interior of the receptacle for collection therein. As such blood or other body fluid withdrawn from the patient by the applied suction will not enter into the hospital's vacuum system. If desired the receptacle or canister 38 may also include a filter (not shown).

If the PPH system 20 does not include the optional suction regulator 30 but does include the optional on/off selector valve 34, the port 34A of the selector valve will be connected to the distal end 22D of the suction tube 22 of the PPH device 20. If the PPH system does not include the optional on/off selector valve 34, but does include the optional suction regulator 30, the tube 40 from the receptacle or canister 38 will be connected to the port 30B of the suction regulator. If the PPH system does not include either the optional suction regulator 30 or the optional on/off selector valve 34, the tube 40 from the canister or receptacle 38 will be connected to the distal end 22D of the suction tube 22 of the PPH device 20.

As best seen in FIG. 6, the intrauterine tip 24 is a rounded member which is formed of a low durometer silicone or other medical-grade elastomeric material, which is atraumatic, e.g., serves to cushion and protect the interior wall of the uterus (e.g., the endometrium, perimetrium or myometrium) during insertion of the distal end of the suction tube therein. For example, the intrauterine tip will act to protect the uterine fundus from injury if the distal end of the suction tube 22 of the PPH device 20 is over-inserted when placed into the uterus.

Turning now to FIGS. 3 and 7 the details of the suction tube 22 and its apertures 26 forming the array of apertures will now be described. The suction tube is a flexible tubular member having a sidewall 22A formed of any biocompatible flexible material, e.g., silicone. The suction tube has of a length of at least 40 cm, and an outside diameter in the range of 6 mm to 16 mm, with a most preferred outside diameter being 1.2 cm. The inside diameter of the central passageway 22B is preferably with the range of 0.4 cm to 1.0 cm, with a most preferably inside diameter of 6 mm. The apertures 26 are each of a constant diameter along their entire length, and are small in size, e.g., a diameter in a preferred range of 1.3 mm to 3.8 mm (with the most preferred diameter being 2 mm).

The apertures 26 are arranged in a symmetrical array of sequentially located groups of apertures, so that the array effectively covers the entire periphery of the distal end portion of the suction tube adjacent the distal end thereof. Preferably the length of the array of apertures is within the range of 5.4 cm to 16.2 cm, with a most preferred length being 10.8 cm. Each group of apertures is composed of plural apertures 26 which extend outward radially through the sidewall 22A of the suction tube 22 from a central longitudinal axis X (FIGS. 3 and 7) which extends down the center of the passageway 22B. In the exemplary embodiment each group of apertures includes four radially extending apertures 26 which are disposed perpendicularly to one another. The groups of apertures are sequentially arranged so that they are angularly offset from each other. In the exemplary embodiment shown the apertures of one group are disposed angularly offset an eighth of a turn (i.e., 45 degrees) about the central longitudinal axis X from the immediately adjacent group of apertures as clearly shown in FIG. 7. The longitudinal spacing between each group of apertures is approximately 6.4 mm.

It should be pointed out at this juncture that the array of apertures and the size, spacing and orientation of the apertures in the array as discussed above is exemplary and other arrangements of apertures are contemplated. Thus, for example, each group of apertures may include at least three apertures and may include more than four apertures. Moreover, the groups of apertures may be offset from one another by more or less than 45 degrees. Further still, the size and spacing of the apertures and the length of the array of aperture may be more or less than the exemplary embodiment shown. That said, it is preferable that for a suction tube 22 having an outside diameter of 12 mm, and a central passageway having an inside diameter of 5.6 mm, the total area of the apertures should be in the range of 10.2 cm² to 29.2 cm². Further still, the apertures 26 need not be of a constant diameter. In fact, it may be desirable to have apertures in the distal end portion of the suction tube that are not the same size or shape as the apertures in more proximally located portion of the suction tube. Moreover, it may be desirable to make use of apertures that are flared so that the diameter of each of the apertures on the outer surface of the sidewall 22A of the suction tube is larger than the diameter of the apertures where they merge with the central passageway 22B.

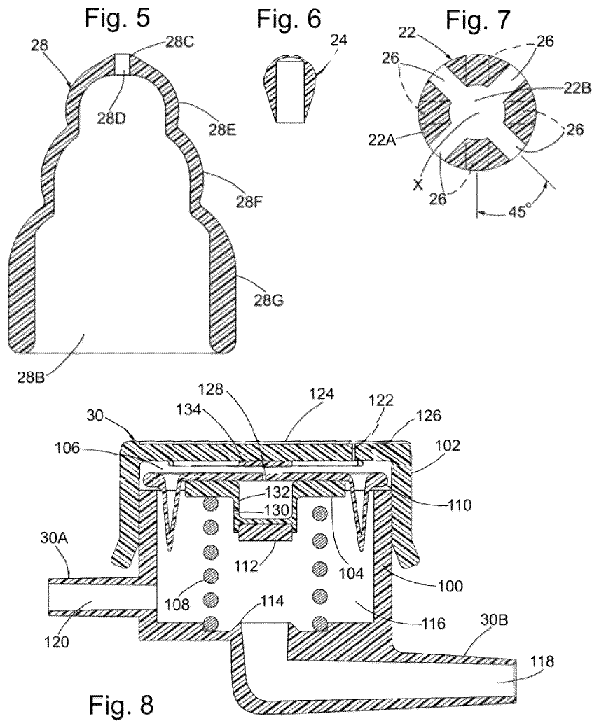

Turning now to FIGS. 3 and 5, the details of the sealing member 28 will now be described. In particular, the sealing member is a generally conically shaped member having a sidewall 28A bounding a hollow interior space 28B, an apex 28C at a top of the sidewall and a generally conically shaped exterior surface. Preferably the sealing member is somewhat flexible for reasons to be described later but need not be flexible. The apex 28C has a central opening 28D, whose inside diameter is approximately the same as the outside diameter of the sidewall 22A of the suction tube, whereupon the central opening is configured to receive the suction tube therein. The exterior surface of the sealing member 28 includes plural, e.g., three, tiers, namely a first tier 28E, a second tier 28F, and a third tier 28G. The first tier has a top and a bottom, with the top forming the apex 28c. The generally conically shaped exterior surface of the first tier is arcuate and has an outside diameter adjacent the apex which is less than the outside diameter of the generally conically shaped exterior surface at the bottom of the first tier. In the exemplary embodiment the outside diameter of the first tier at the apex is approximately 0.8 cm, and the outside diameter of the bottom of the first tier is approximately 4.8 cm. The second tier 28F also has a top and a bottom, with the top of the second-tier merging with the bottom of the first tier. The generally conically shaped exterior surface of the second tier is also arcuate and has an outside diameter adjacent the top of the second tier which is less than the outside diameter of the generally conically shaped exterior surface at a bottom of the second tier. In the exemplary embodiment the outside diameter of the second tier at its top is approximately 4.8 cm, and the outside diameter at its bottom is approximately 6.6 cm. The third tier 28G also has a top and a bottom, with the top of the third tier merging with the bottom of said second tier. The generally conically shaped exterior surface of the third tier is also arcuate and has an outside diameter adjacent the top of the third tier which is less than the outside diameter of the generally conically shaped exterior surface at a bottom of the third tier. In the exemplary embodiment the outside diameter of the third tier at its top is approximately 6.6 cm, and the outside diameter at its bottom is approximately 8.6 cm. It should be noted at this juncture that the foregoing dimensions are merely exemplary and other dimensions are contemplated. For example, the first tier may have a maximum external diameter in the range of 2 to 6 cm, the second tier may have a maximum external diameter in the range of 3 to 9 cm, and the third tier may have a maximum external diameter in the range of 5 to 14 cm.

The sealing member 28 being somewhat flexible is preferably formed of any suitable generally flexible, biocompatible material, e.g., silicone, so that it can engage the anatomy of the patient and flex, if necessary, to form the desired seal to effectively maintain negative pressure within the uterus, without causing discomfort or injury to the patient.

Turning now to FIG. 8 the details of the optional suction regulator 30 will now be described. To that end, as can be seen the suction regulator 30 is housed within a hollow housing 100 which includes a cover 102. The housing and the cover are each formed of a rigid plastic, such as ABS. A piston 104 is disposed within the housing and is also formed of a rigid plastic, such as ABS. The space between the upper surface of the piston and the cover forms an upper chamber 106 that is at the pressure of the ambient atmosphere. A helical spring 108 is located within the housing under the piston and serves to bias the piston upward in the housing. The spring is formed of any suitable material, e.g., stainless steel. A rolling diaphragm 110 is coupled to the piston and the housing. In particular, the diaphragm 110 is engaged in a sealing fashion between the housing 100 and the cover 102. The diaphragm can be formed of any suitable material, e.g., Nitrile. A seal 112 is located on the underside of the piston and is arranged to engage a seat 114 when excess suction is applied (as will be described later). The seal 112 is formed of any suitable material, e.g., silicone rubber. The interior of the housing just below the cover 104 and above diaphragm 110 forms the heretofore identified first chamber 106, while the portion of the housing below the diaphragm forms a second or lower chamber 116, with the diaphragm isolating the two chambers from each other. The port 30B, which as discussed earlier is configured to receive the suction from the vacuum source 32 is in the form of a conduit extending from the housing 100 and having a passageway 118 extending therethrough. The seat 114 extends about the periphery at the inner end of the passageway 118 and is in the form of a beveled upstanding wall. The port 30A constitutes a conduit in the form of a tubular extension from the housing 100 and has a passageway 120 extending therethrough. The passageway 120 serves to carry the regulated suction from the suction regulator 30 to the suction tube 22 of the PPH device 20, and from thence to the interior of the patient's uterus.

Operation of the suction regulator 30 is as follows. Suction is applied from the vacuum source 32 through the open on/off selector valve 34 and into the passageway 118 of conduit forming the port 30B at the suction side of the regulator. The suction regulator is configured to maintain the amount of suction applied to the patient to a predetermined, desired level, even if a suction level greater than the predetermined level is applied to it via the port 30B and its passageway 118 from the vacuum source 32. The predetermined or desired suction level is established by the spring 108 and dimensions of the housing 100, the piston 104 and the seal 112. In this regard the pressure within the upper chamber 206 will be equal to atmospheric pressure by virtue of the communication of that chamber with the ambient atmosphere via a vent hole 122 in the cover 102. With suction applied, the pressure differential between the chambers 106 and 116 forces the diaphragm and the piston downward toward the seat 114 of the housing against the bias of the spring 108. If the suction applied via passageway 118 of the port 30B is greater that the predetermined level the piston and diaphragm will move such that the seal 112 on the bottom of the piston comes into engagement with the seat 114 thereby isolating the lower chamber 116 from the vacuum source 32. This action thereby limits the level of suction in chamber 116 and ultimately in the patient's uterus to the predetermined (regulated) level. If, however, the suction applied via port 30B is less than the predetermined level, the piston and diaphragm will only move part of the way downward. As such, the level of suction applied to the patient via the passageway 120 in the port 30A will equal that in port 30B so that the level of suction at the port 30B will be applied to the uterus of the patient.

As can be seen in FIGS. 4 and 8, the cover 102 includes the heretofore mentioned vent hole 122, which will be referred to hereinafter as the atmospheric reference port. That port 122 extends through the thickness of the cover and is in fluid communication with the interior of upper chamber 106 to maintain that chamber at atmospheric pressure. Inasmuch as the atmospheric reference port 122 is located in the top surface of the cover, it is susceptible to being blocked or covered by a sticker, some other object or even the finger of a user. To prevent such an occurrence the cover 102 includes a thickened portion 124 located adjacent the atmospheric reference port 122, with an elongated shallow slot 126 (FIG. 4) extending through the thickened portion. The outer or top end of the atmospheric reference port is located at the bottom of the slot 126 and is in fluid communication therewith. Each end of the slot is open. Thus, if something should be on the top surface of the thickened portion of the cover disposed over the atmospheric reference port air can still enter into that port via any open end of the slot.

If the suction source 32 is operated in a manner such that a high level of suction is applied very rapidly, the piston 104 may experience an over-travel, wherein it moves downward very quickly such that the seal 112 on the underside of the piston may become stuck on the seat 114. Under this condition the suction applied to the patient's uterus would have a higher level of vacuum than the suction regulator was configured to provide. The suction regulator could stay in that state for an extended and indefinite period of time, particularly if the apertures 26 in the suction tube 22 are blocked. To prevent such an occurrence, the suction regulator 30 includes two bleed holes 128 and 130. In particular, the diaphragm 110 includes a bleed hole 128 in the central portion thereof. The piston 104 includes a hollow portion defined by a side wall 132. The sidewall 132 includes a bleed hole 130 extending radially therethrough. Thus, the interior of the hollow portion of the piston is in fluid communication with the lower chamber 116 via the bleed hole 130. Since the diaphragm 110 includes the bleed hole 128, which is in communication with the upper chamber 106, that chamber will be in fluid communication with the lower chamber via the bleed hole 128. Hence, if the piston should become stuck on the seat 114, air which enters into the upper chamber 106 via the atmospheric reference port 122 can then pass through the bleed hole 128 into the interior of the piston and from there through the bleed hole 130 into the lower chamber 116. The ingress of air into the lower chamber will decrease the vacuum within that chamber, thus enabling the spring 108 to move the piston 104 upward so that the seal 112 is off of the seat 114. In accordance with one exemplary preferred embodiment of the suction regulator 30, the bleed hole 128 is approximately 0.1 inch in diameter, while the bleed hole 130 is approximately 0.015 inch in diameter. During typical operation the flow rate of air into chamber 116 via the bleed hole 130 is in the range of 10 standard cubic feet per hour (SCFH) or lower. The underside of the cover 102 includes a disk 134 located thereon. The disk 134 is formed of the same material as the material of the seal 112. The disk 134 serves to seal the diaphragm's bleed hole 128 when the piston 104 is in its maximum up position to prevent the ingress of fluid into the lower chamber 116.

The methods of use of the PPH device 20 will now be described. For example, in accordance with one method of use, and as best seen in FIG. 1A the PPH device 20 is located within the female patient in the following manner. The intrauterine tip 24 is gently inserted through the patient's vagina 2 and cervix 4. The suction tube 22 is advanced until its distal end portion with the array of apertures 26 is located fully within the patient's uterus 9. The sealing member 28 is then used to continue insertion of the suction tube until resistance is encountered from the walls of the vagina contacting the outer surface of the sealing member 28. The atraumatic intrauterine tip 24 serves to prevent any injury to the uterine wall if the suction tube is attempted to be inserted too far into the uterus. Once the distal portion of the suction tube 22 is fully inserted into the uterus and secured in position by the sealing member 28, the proximal portion of the suction tube exiting from the base of the sealing member 28 is connected a receptacle or canister 38 which is then connected to a vacuum source as shown in FIG. 1A. The PPH device 20 is now ready to apply suction to the patient's uterus upon opening of the line from the vacuum source. Once activated, suction is delivered through the plural radially extending apertures 26 in all directions into the interior of the uterus, whereupon the negative pressure of the applied suction will induce uterine contraction to counteract uterine atony to effect the lessening or termination of postpartum hemorrhaging. To that end, if the PPH system includes the optional suction regulator 30 and optional on/off selector valve 34, once suction is activated, the slide 34C of the valve 34 is slid from the closed or off position to the open or on position, whereupon the suction from the vacuum source 32 is routed through the suction regulator 30. The suction regulator operates automatically to reduce the level of suction to the desired operating or regulated level, e.g., 80 mmHg+/−10 mmHg, and delivers the regulated suction into the distal end of the central passageway of the suction tube.

It should be noted that the closure formed by the engagement of the sealing member with the walls of the vagina need not be fully hermetic, but merely sufficient to secure the device in place and prevent enough ambient air from entering into the vagina and into the cervix, which could interfere or reduce the negative pressure in the uterus.

Once the suction applied to the interior of the uterus, to effect sufficient lessening or termination of postpartum hemorrhage, the delivery of suction can be discontinued. If the optional suction regulator 30 and optional on/off selector valve 34 is used, slide 34C of the on/off selector valve 34 can then be slid to the off position, thereby isolating the vacuum source 32 from the suction regulator 30, whereupon suction will no longer be applied into the interior of the uterus. Once suction has been discontinued, the suction tube 22 of the PPH device 20 can then be withdrawn from the patient's uterus by flexing the sealing member to loosen it from the vaginal wall. That action enables the suction tube to be easily withdrawn from the patient's body until it is fully outside the patient's body.

As should be appreciated by those skilled in the art since the sealing member includes a pliable sidewall which is tiered in plural arcuate sections which together form a somewhat undulated conical shaped hollow member the sealing member can accommodate variations in vaginal size and can be located at various positions with respect to the patient's vagina so that the sealing member effectively seals the vagina from the ambient atmosphere, whereupon suction applied by the suction tube within the uterus effectively contracts the uterus to lessen or terminate postpartum hemorrhage. Thus, the PPH device 20 is quite accommodating and very easy to use, e.g., it does not require precise positioning of an inflatable balloon at the external os of the cervix, as does the Jada® system.

Turning now to FIG. 1B, the PPH system like that of FIG. 1A is shown during an alternative method of use, wherein the tier section 28E of the sealing member 28 contiguous with its apex 28C has been slid to a position located within the vagina, with the underlying tiered section 28F also being located within the vagina. Once the suction tube 22 and the sealing member 28 are in the position shown in FIG. 1B, the PPH device can be operated in the same manner as described with respect to FIG. 1A.

Turning now to FIG. 1C, the PPH system like that of FIG. 1A is shown during another alternative method of use, wherein the entire sealing member 28 is located within the vagina with its apex is located adjacent the cervix. Once the suction tube 22 and the sealing member 28 are in the position shown in FIG. 1C, the PPH device can be operated in the same manner as described with respect to FIG. 1A.

As should be appreciated by those skilled in the art, since the sealing member is preferably flexible, it enables a physician or other health care provider to flex or otherwise depress the proximal end portion of the side wall when the sealing member is in place to provide access to anatomy which would otherwise have been obscured by the sealing member. Thus, for example, if the vaginal wall has a tear or other defect which the physician wishes to address, the proximal portion of the sidewall of the sealing member can be shifted or otherwise adjusted to expose that tear or other injury, whereupon it can be sutured or otherwise repaired. Moreover, providing the sealing member with flexibility enables the physician or other health care provider to deform the proximal portion of the sealing member's sidewall between his/her thumb and index finger to facilitate the removal of the device from the patient.

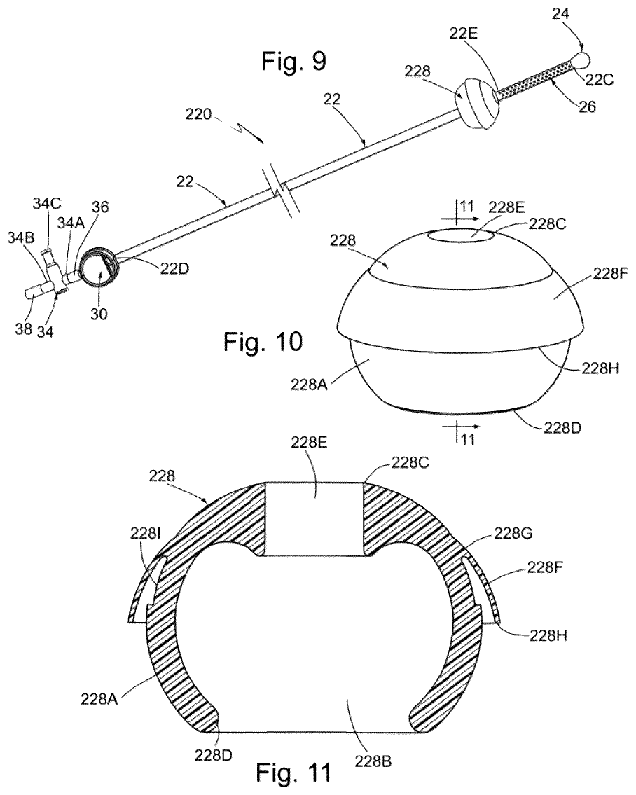

Turning now to FIGS. 9-12, there is shown an alternative embodiment of a PPH device 220 constructed in accordance with this invention. The PPH device 220 is identical in construction and use as the PPH device 20, except that its sealing member 228 is different than the sealing member 28. The details of the construction and operation of the sealing member 228 will be described shortly. In the interest of brevity, the common features of the sealing member 228 with the sealing member 28, will be given the same reference numbers and the details of the construction and operation of the PPH device 220 will not be reiterated.

Thus, as best seen in FIGS. 10 and 11 the sealing member 228 is a bulbous member having a sidewall 228A bounding a hollow interior space 228B, an apex 228C at a top of the sidewall and a generally partially spherically shaped exterior surface terminating in a proximal end or bottom edge 228D. The bottom edge 228D is rounded. Like the sealing member 28, the sealing member 228 is preferably somewhat flexible, e.g., is formed of silicone or some other biocompatible flexible material but need not be flexible. The apex 228C has a central opening 228E, whose inside diameter is approximately the same as the outside diameter of the sidewall 22A of the suction tube 22, whereupon the central opening is configured to receive the suction tube 22 therein. The exterior surface of the sealing member 228 includes a flexible skirt 228F. The skirt 228F is of a convex arcuate shape. In its normal quiescent state, like shown in FIGS. 9 and 10, the skirt extends outward at an acute angle to the exterior surface from a merger point 228G which is located slightly above the midpoint between the apex and bottom 228D of the sealing member. The skirt has a length of approximately 1.7 cm from the merger point 228G to the proximal free end 228H of the skirt, but that length can be greater or lesser, if desired. What is important is that the skirt has a sufficient length that upon the creation of a vacuum within the uterus resulting from the application of suction thereto via the suction tube 22, the skirt will automatically flex outward or inward from its normal quiescent state to engage the vaginal wall to form a sufficient seal therewith to maintain the vacuum within the uterus. In addition to the skirt flexing, the sidewall of the sealing member may also flex somewhat so that the portion of it located below (proximally) of the skirt may also engage the vagina to enhance the sealing of the uterus from loss of vacuum in the uterus or movement of the patient or suction tube. Moreover, being flexible the bottom (proximal portion) of the sidewall 228A of the sealing member 228 may be squeezed by a physician when the plug is in place in the vagina to provide access to portions of the vagina that the physician may want to address, e.g., to a tear in the vaginal wall the physician will want to suture or otherwise repair or to facilitate removal of the sealing member from the vagina as discussed earlier.

In order to provide additional flexibility for the sidewall 228, the portion of the sidewall from the point 228G includes an optional annular recess 228I in the outer surface thereof. The annular recess 228I renders the sidewall at the location of the annular recess more flexible than the remaining portion of the sidewall. Accordingly, if the physician wishes to flex the sidewall to provide access to a portion of the vagina which would otherwise be covered by the sidewall, all that needs to be done is for the physician to squeeze the bottom of the sidewall between his/her index finger and thumb to flex the sidewall away from the vagina. Moreover, the ability to flex the bottom of the sidewall enables the physician to readily remove the sealing member from within the vagina.

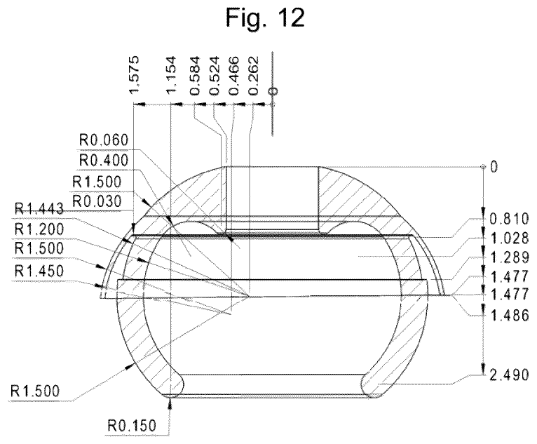

FIG. 12 shows various dimensions of one exemplary sealing member 228 constructed in accordance with this invention, with all dimensions being shown measured in inches.

As mentioned above, the PPH device 220 can be used in the same manner as the method of use of the PPH device 20.

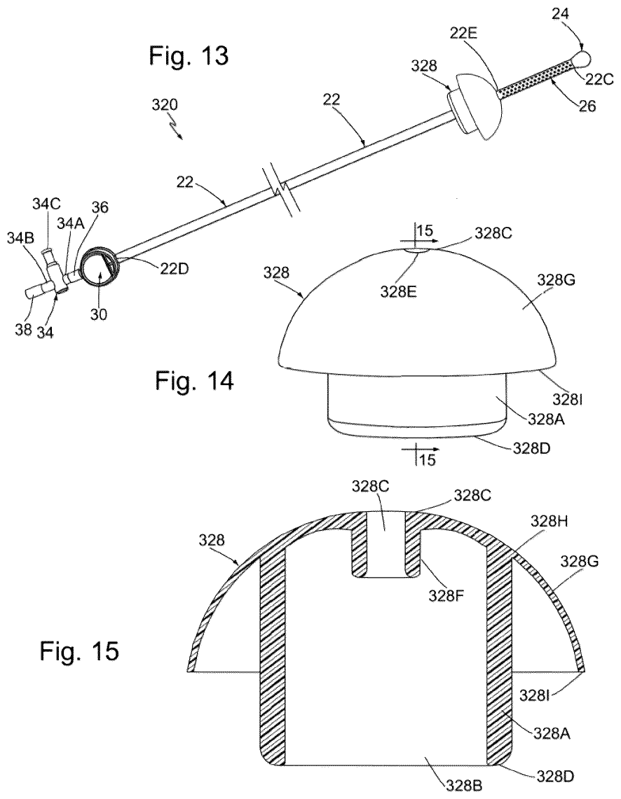

Turning now to FIGS. 13-16, there is shown still another alternative embodiment of a PPH device 320 constructed in accordance with this invention. The PPH device 320 is identical in construction and use as the PPH devices 20 and 220, except that its sealing member 328 is different than the sealing member 28 and 228. The details of the construction and operation of the sealing member 328 will be described shortly. In the interest of brevity, the common features of the sealing member 328 with the sealing members 28 and 228, will be given the same reference numbers and the details of the construction and operation of the PPH device 320 will not be reiterated in the interest of brevity.

Thus, as best seen in FIGS. 14 and 15 the sealing member 328 is a cylindrical member having a sidewall 328A bounding a hollow interior space 328B. The top or distal portion of the sidewall 328A is of arcuate convex shape having an apex 328C at a top thereof. The portion of the sidewall located below the top portion terminates in a proximal end or bottom edge 328D. The bottom edge 328 is rounded. Like the sealing members 28 and 228, the sealing member 328 is preferably somewhat flexible, e.g., is formed of silicone or some other biocompatible flexible material but need not be flexible. The apex 328C has a central opening 328E formed by an annular collar 328F, whose inside diameter is approximately the same as the outside diameter of the sidewall 22A of the suction tube 22, whereupon the central opening 328E is configured to receive the suction tube 22 therein. The exterior surface of the sealing member 328 includes a flexible skirt 328G. The skirt 328G is of an arcuate convex shape and forms a continuous extension of the exterior surface of the top of the sidewall 328A. In its normal quiescent state, like shown in FIGS. 14 and 15, the skirt 328G extends outward at an acute angle to the exterior surface from a merger point 328H which is located at the top of the cylindrical portion of the sidewall 328A. The skirt has a length of approximately 2.4 cm from the merger point 328H to the proximal free end 328I of the skirt, but that length can be greater or lesser, if desired. What is important is that the skirt has a sufficient length that upon the creation of a vacuum within the uterus resulting from the application of suction thereto via the suction tube 22, the skirt will automatically flex outward or inward from its normal quiescent state to engage the vaginal wall to form a sufficient seal therewith to maintain the vacuum within the uterus. In addition to the skirt flexing, the sidewall of the sealing member may also flex somewhat so that the portion of it located below (proximally) of the skirt may also engage the vagina to enhance the sealing of the uterus from loss of vacuum in the uterus. Moreover, being flexible the bottom (proximal portion) of the sidewall 328A of the sealing member 328 may be squeezed by a physician when the plug is in place in the vagina to provide access to portions of the vagina that the physician may want to address, e.g., to a tear in the vaginal wall the physician will want to suture or otherwise repair or to facilitate removal of the sealing member from the vagina as discussed earlier. Accordingly, if the physician wishes to flex the sidewall to provide access to a portion of the vagina which would otherwise be covered by the sidewall, all that needs to be done is for the physician to squeeze the bottom of the sidewall between his/her index finger and thumb to flex the sidewall away from the vagina. Moreover, the ability to flex the bottom of the sidewall enables the physician to readily remove the sealing member 328 from within the vagina.

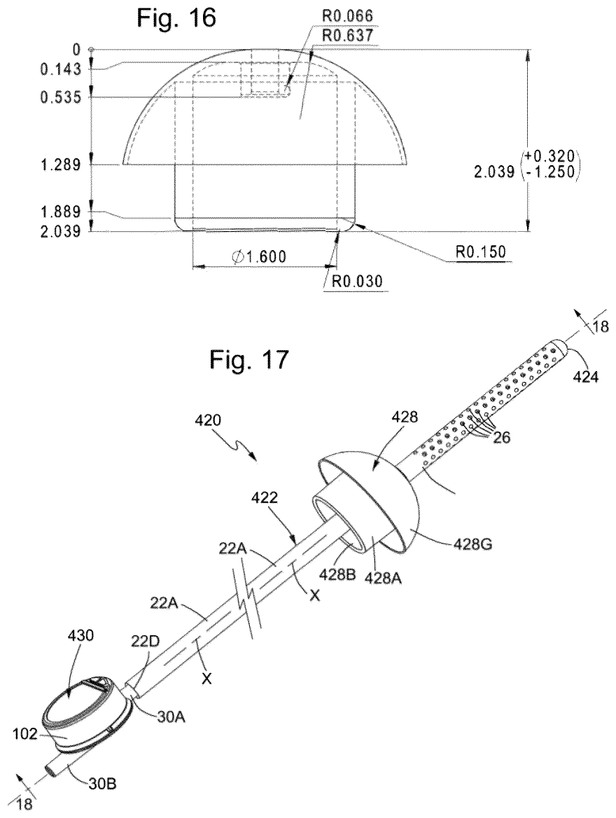

FIG. 16 shows various dimensions of one exemplary sealing member 328 constructed in accordance with this invention, with all dimensions being shown measured in inches.

The PPH device 320 can be used in the same manner as the method of use of the PPH devices 20 and 220.

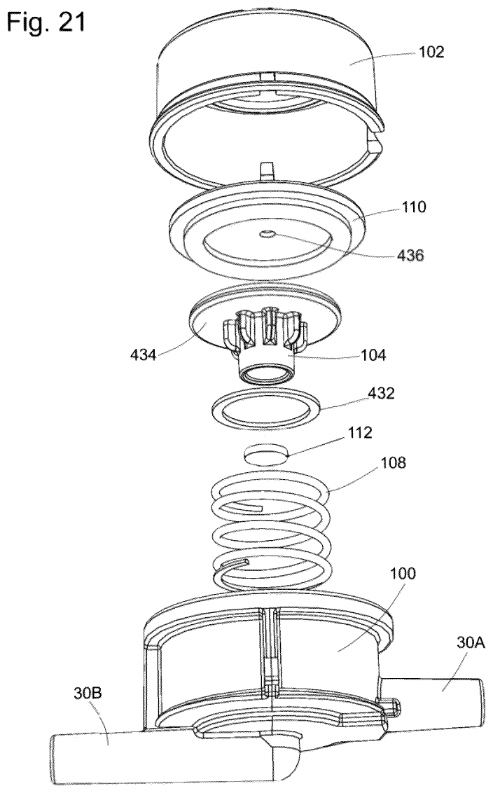

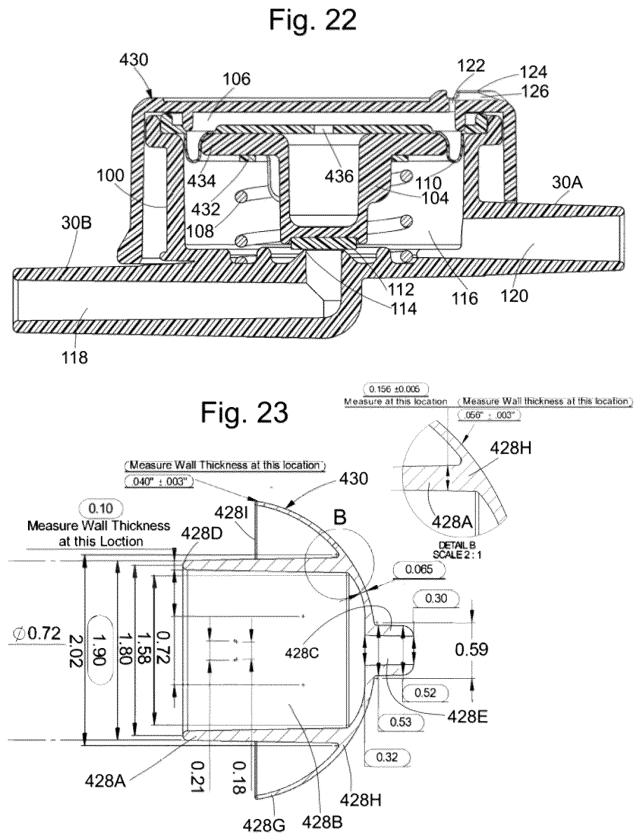

Turning now to FIGS. 17-23, there is shown still another alternative embodiment of a PPH device 420 constructed in accordance with this invention. The PPH device 420 can be used in the same manner as the method of use of the PPH devices 20, 220 and 320.

The PPH device 420 is identical in construction and use as the PPH devices 20, 220, and 320 except that its suction tube 422 is slightly different than the suction tube 22. In addition, the sealing member 428 of the PPH device 420 is somewhat different than the sealing members 28, 228 and 328 described above. The PPH device 420 also includes an optional suction regulator 430, which is also slightly different in construction to the suction regulator 30 described above. In the interest of brevity the common features of the suction tube 422 with the suction tube 22, will be given the same reference numbers and the details of its construction and operation will not be reiterated. So too, the common features of the sealing member 428 with the sealing members 28, 228 and 328, will be given the same reference numbers and the details of its construction and operation will not be reiterated. Further still, the common features of the suction regulator 430 with the suction regulator 30 will be given the same reference numbers and the details of its construction and operation will not be reiterated.

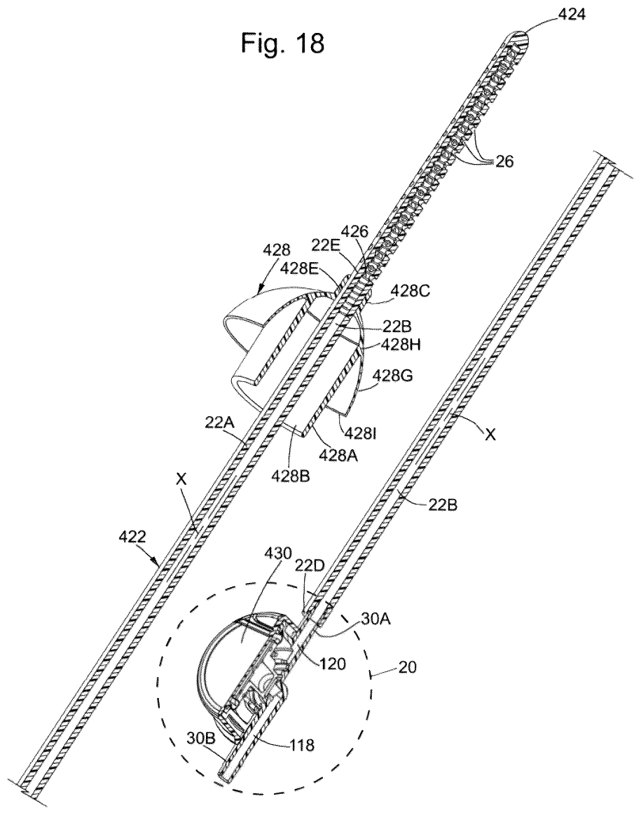

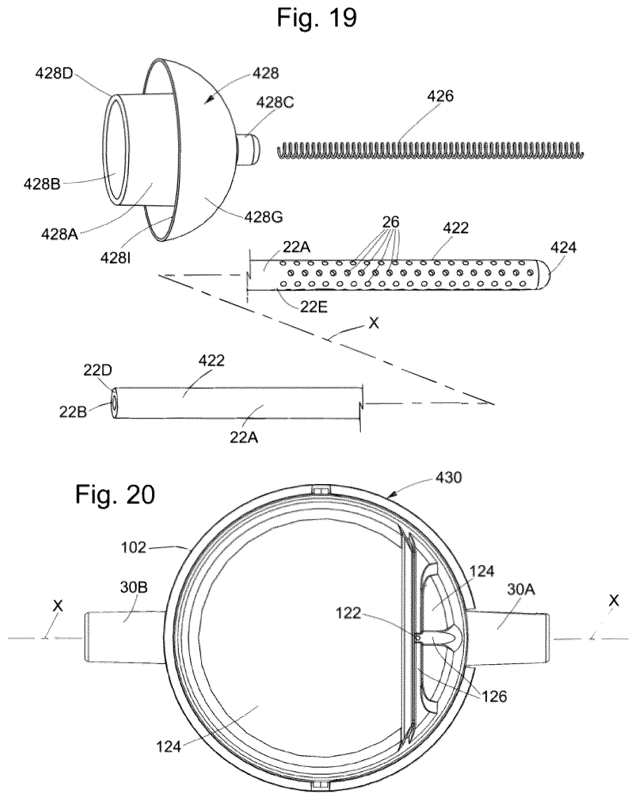

Thus, as best seen in FIGS. 17 and 18 the suction tube 422 has a sidewall 22A which bounds a central longitudinally extending passageway 22B. The distal end of the suction tube is closed and rounded to form an intrauterine tip 424, which is atraumatic. The atraumatic tip closes off the distal end of the central passageway 22B. Like the suction tube 22 described above, the suction tube 422 includes a plurality of radially extending apertures or holes 26 located in an array extending through the sidewall 22A of the suction tube from its atraumatic tip to an intermediate point 22E, e.g., approximately 4 cm to 14 cm proximally of the atraumatic tip. Each of the apertures or holes 26 is in fluid communication with the central passageway 22B of the suction tube. The proximal end 22D of the suction tube 422 is open and configured to receive the port 30A of the optional suction regulator 430. An optional helical spring 426 which is best seen in FIG. 19 is disposed within the central passageway 22B and extends from the distal end of the passageway 22B and may or may not extend the entire length of the array of apertures 26. The spring is approximately 40 French and preferably formed of stainless steel. The spring serves to provide additional structural integrity to facilitate insertion of the device.

Turning now to FIGS. 17-19 and 23, the details of the sealing member 428 will now be described. The sealing member 428 is a cylindrical member having a sidewall 428A bounding a hollow interior space 428B. The top or distal portion of the sidewall 428A is of arcuate convex shape having a tubular nipple 428C at the apex thereof. The portion of the sidewall located below the top portion terminates in a proximal end or bottom edge 428D. Like the sealing members 28, 228 and 328, the sealing member 428 is preferably somewhat flexible, e.g., is formed of silicone or some other biocompatible flexible material but need not be flexible. The nipple 428C has a central opening 428E, whose inside diameter is approximately the same as the outside diameter of the sidewall 22A of the suction tube 422, whereupon the central opening 428E is configured to frictionally receive the suction tube 22 therein. The exterior surface of the sealing member 428 includes a flexible skirt 428G. The skirt 328G is of an arcuate convex shape and forms a continuous extension of the exterior surface of the top of the sidewall 428A. In its normal quiescent state, like shown in FIGS. 17-19 and 23, the skirt 428G extends outward at an acute angle to the exterior surface from a merger point 428H which is located at the top of the cylindrical portion of the sidewall 428A.

The passageway 428E is configured to frictionally receive the suction tube 422 so that the sealing member 428 can be slid along the suction tube to a desired position within the vagina of the patient in a similar manner to the positioning of the sealing members 28, 228 and 328. In particular, the use of the PPH device 420 is as follows: The intrauterine atraumatic tip 424 is gently inserted through the patient's vagina 2 and cervix 4. The suction tube 422 is advanced until its distal end portion with the array of apertures 26 is located fully within the patient's uterus 9. The atraumatic intrauterine tip 424 serves to prevent any injury to the uterine wall if the suction tube is attempted to be inserted too far into the uterus. The sealing member 428 is then slid along the suction tube until resistance is encountered from the walls of the vagina contacting the outer surface of the sealing member. Once the distal portion of the suction tube 422 is fully inserted into the uterus and the sealing member has been slid along the suction tube to its desired position thereon the resistance between the vaginal tissue and the sealing member will deter accidental movement or displacement of the sealing member from that desired position on the suction tube.

With the PPH device now secured in position by the sealing member 428, the proximal portion of the suction tube which is connected to the suction regulator 430 is then connected to the receptacle or canister 38 which is then connected to a vacuum source as described previously. The PPH device 420 is now ready to apply regulated suction to the patient's uterus. In particular, the regulated suction from the optional suction regulator 430 is delivered through the plural radially extending apertures 26 in the suction tube 422 in all directions into the interior of the uterus, whereupon the negative pressure of the applied suction will induce uterine contraction to counteract uterine atony to effect the lessening or termination of postpartum hemorrhaging.

As should be appreciated by those skilled in the art, the skirt 428G of the sealing member 428, like the skirt 328G of the sealing member 328, has a sufficient length that upon the creation of a vacuum within the uterus resulting from the application of suction thereto via the suction tube 422, the skirt will automatically flex outward or inward from its normal quiescent state to engage the vaginal wall to form a sufficient seal therewith to maintain the vacuum within the uterus. In addition to the skirt flexing, the sidewall of the sealing member may also flex somewhat so that the portion of it located below (proximally) of the skirt may also engage the vagina to enhance the sealing of the uterus from loss of vacuum in the uterus. Moreover, being flexible the bottom (proximal portion) of the sidewall 428A of the sealing member 428 may be squeezed by a physician when the plug is in place in the vagina to provide access to portions of the vagina that the physician may want to address, e.g., to a tear in the vaginal wall the physician will want to suture or otherwise repair or to facilitate removal of the sealing member from the vagina as discussed earlier. Accordingly, if the physician wishes to flex the sidewall to provide access to a portion of the vagina which would otherwise be covered by the sidewall, all that needs to be done is for the physician to squeeze the bottom of the sidewall between his/her index finger and thumb to flex the sidewall away from the vagina. Moreover, the ability to flex the bottom of the sidewall enables the physician to readily remove the sealing member 428 from within the vagina.

FIG. 23 shows various dimensions of the exemplary sealing member 428 constructed in accordance with this invention, with all dimensions being shown measured in inches.

Turning now to FIGS. 20-22 the details of the optional suction regulator 430 will now be described. The suction regulator 430 is similar in many respects to the suction regulator 30 described heretofore. Thus, in the interest of brevity the common components or features of the suction regulator 430 with the suction regulator 30 will be given the same reference numbers and the details of their construction, arrangement and operation will not be reiterated. Accordingly, as can be seen the suction regulator 430 is housed within a hollow housing 100 which includes a cover 102. A piston 104 is disposed within the housing. The space between the upper surface of the piston and the cover forms an upper chamber 106 that is at the pressure of the ambient atmosphere. A helical spring 108 is located within the housing under the piston and serves to bias the piston upward in the housing. A rolling diaphragm 110 is coupled to the piston and the housing. The diaphragm 110 is engaged in a sealing fashion between the housing 100 and the cover 102. A seal 112 is located on the underside of the piston and is arranged to engage a seat 114 in the housing when excess suction is applied. The interior of the housing just below the cover 104 and above diaphragm 110 forms the heretofore identified upper chamber 106, while the portion of the housing below the diaphragm forms another or lower chamber 116, with the diaphragm isolating the two chambers from each other. The port 30B, which as discussed earlier is configured to receive the line suction from the vacuum source 32 is in the form of a conduit extending from the housing 100 and having a passageway 118 extending therethrough. The seat 114 extends about the periphery at the inner end of the passageway 118 and is in the form of a beveled upstanding wall. The port 30A constitutes a conduit in the form of a tubular extension from the housing 100 and has a passageway 120 extending therethrough. The passageway 120 serves to carry the regulated suction from the suction regulator 30 to the suction tube 22 of the PPH device 420, and from thence to the interior of the patient's uterus.

A regulator shim 432 in the form of a generally planar ring formed of stainless steel is located within the chamber 116 immediately below the flanged top portion 434 of the piston.

Operation of the suction regulator 430 is as follows. Suction is applied from the vacuum source 32 through the open on/off selector valve 34 and into the passageway 118 of conduit forming the port 30B at the suction side of the regulator 430. The suction regulator is configured to limit the amount of suction applied to the patient to a predetermined, desired level, even if a suction level greater than the predetermined level is applied to it via the port 30B and its passageway 118 from the vacuum source 32. The predetermined or desired suction level is established by the spring 108 and dimensions of the housing 100, the piston 104 and the seal 112. In this regard the pressure within the upper chamber 206 will be equal to atmospheric pressure by virtue of the communication of that chamber with the ambient atmosphere via an atmospheric reference port or vent hole 122 in the cover 102. With suction applied, the pressure differential between the chambers 106 and 116 forces the diaphragm and the piston downward toward the seat 114 of the housing against the bias of the spring 108. If the suction applied via passageway 118 of the port 30B is greater that the predetermined level the piston and diaphragm will move such that the seal 112 on the bottom of the piston comes into engagement with the seat 114 thereby isolating the lower chamber 116 from the vacuum source 32. This action thereby limits the level of suction in chamber 116 and ultimately in the patient's uterus to the predetermined (regulated) level. If, however, the suction applied via port 30B is less than the predetermined level, the piston and diaphragm will only move part of the way downward. As such, the level of suction applied to the patient via the passageway 120 in the port 30A will equal that in port 30B so that the level of suction at the port 30B will be applied to the uterus of the patient.

In order to prevent the atmospheric reference port 122 from being blocked or covered, which could impede proper operation of the suction regulator 430, the cover 102 includes a thickened portion 124 located adjacent the atmospheric reference port 122, with an elongated shallow slot 126 extending through the thickened portion. The outer or top end of the atmospheric reference port is located at the bottom of the slot 126 and is in fluid communication therewith. Each end of the slot is open. Thus, if something should be on the top surface of the thickened portion of the cover disposed over the atmospheric reference port air can still enter into that port via any open end of the slot.

The diaphragm 110 includes a hole 436 in the central portion thereof. The hole is approximately 0.36 cm in diameter and serves to improve the response time of the regulator by allowing a small amount of air to flow through thereby having an equalizing effect when the suction supplied by the vacuum source is considerably higher than the set point.

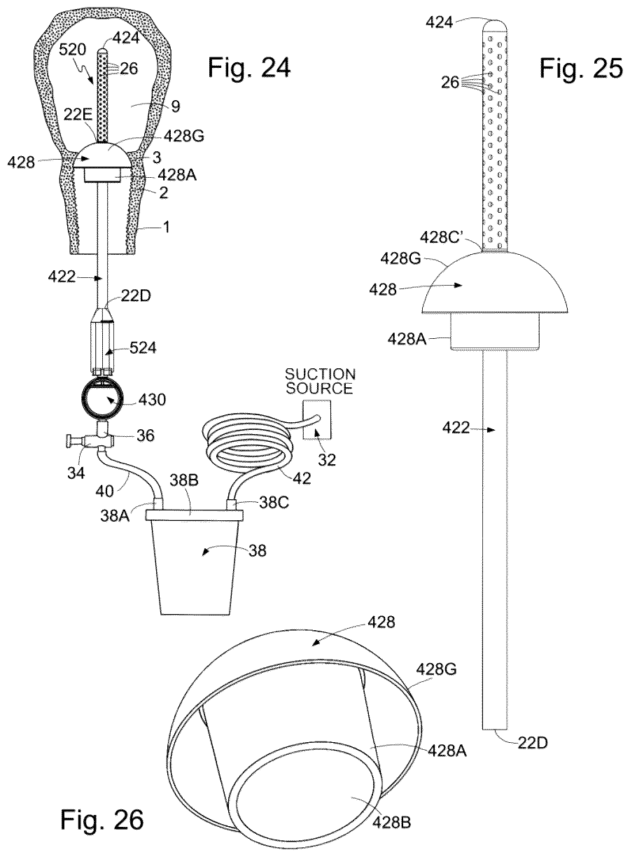

Turning now to FIGS. 24-39, there is shown still another alternative embodiment of a PPH device 520 constructed in accordance with this invention. The PPH device 520 is similar in construction and use as the PPH device 420, except for a minor change in the construction of the sealing member 428, and a major change in the suction tube 422. The change in the suction tube constitutes the inclusion of an illumination system 524 in the suction tube 422 for illuminating portions of the patient's anatomy into which the PPH device 520 is placed to facilitate that placement, as will be described later.

In the interest of brevity, the common features of the PPH device 520 with the PPH device 420 will be given the same reference numbers and the details of the construction and the operation of the common features of those devices will not be reiterated. The PPH device 520 can be used in the same manner as described above with respect to FIGS. 1A, 1B, and 1C by connecting it to the receptacle or canister 38 and the suction source 32 via the tubing sections 40 and 42 as shown therein.

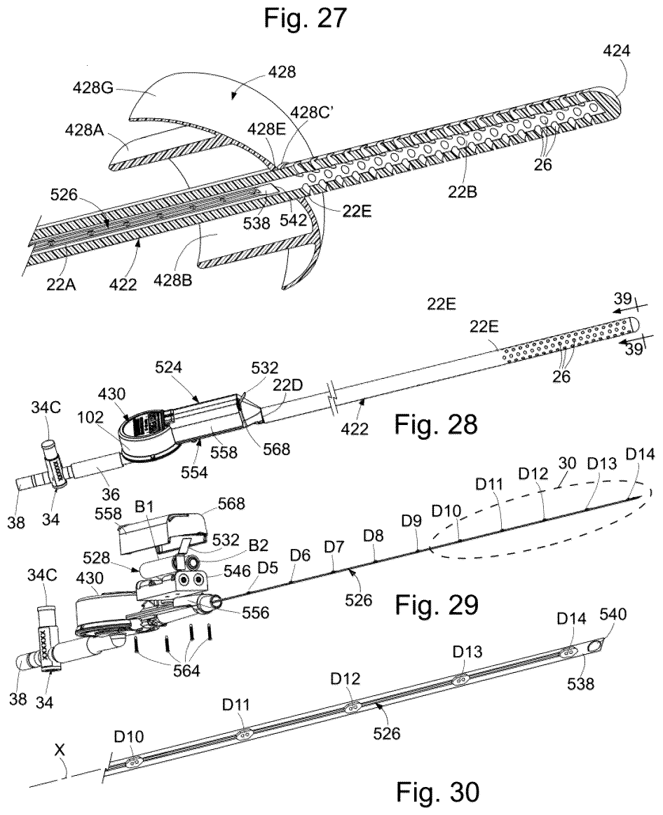

The minor change in the sealing member 428 is the fact that the nipple 428C has been shortened in length and is now designated by the reference number 428C′. As can be seen best in FIG. 27 the shortened nipple 428C′ includes the central opening 428E, whose inside diameter is approximately the same as the outside diameter of the sidewall 22A of the suction tube 422. The the central opening 428E is configured to frictionally receive the suction tube 22 therein so that the position of the sealing member 428 can be adjusted as desired to the particular anatomy of the patient so that the apertured distal end portion of the suction tube is located within the uterus like shown in FIG. 24.

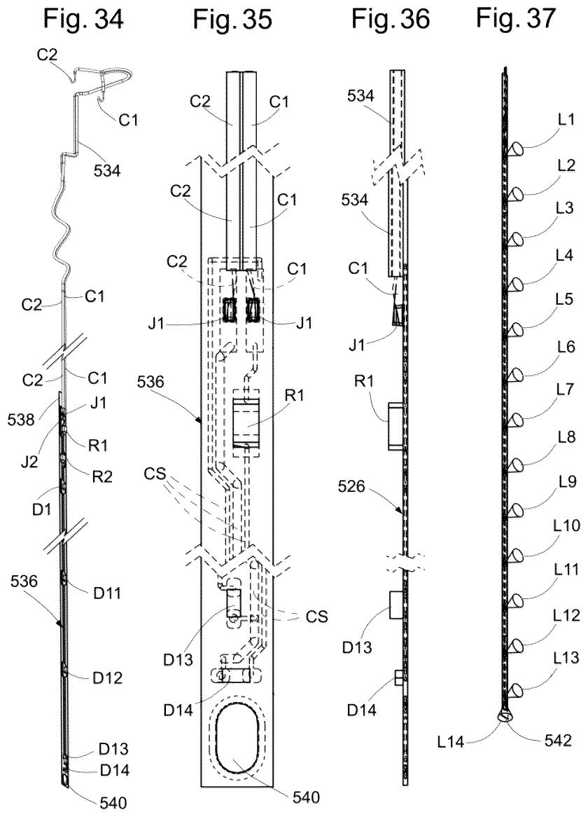

The illumination system 524 is best seen in FIGS. 28 and 29 and basically comprises a lighting subsystem 526 and an electrical power source 528. The lighting subsystem 526 includes an elongated source of illumination (also referred to herein as an elongated light source) composed of a series of visible light emitting diodes LEDs D1-D14 located within the central passageway 22A of the suction tube 422. The illumination system 524 also includes an electrically conductive cable 534 (FIG. 34) and an encasement sleeve 538 (FIG. 32) in which the elongated light source is located. The electrical power source 528 constitutes an assembly which is mounted within a housing (to be described later) on a portion of the suction regulator 430. The electrical power source 528 is connected to the elongated light source 526 by the electrically conductive cable 534 and provides electrical power to it. That action causes the elongated light source 526 to produce visible light. The elongated light source 526 is located in the central passageway 22A in the portion of the suction tube that is located on the proximally located side of the sealing member 428. The suction tube is preferably transparent or translucent so that the visible light produced by the LEDs D1-D14 will pass through the sidewall of the suction tube 422 to illuminate the portion of the patient's body, e.g., the patient's vagina, through which that portion of the suction tube extends, as shown in FIG. 24.

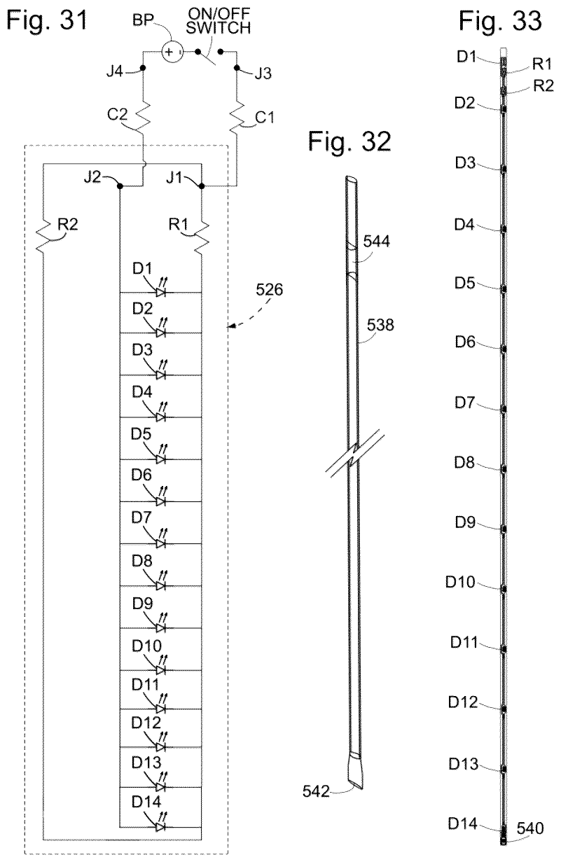

In accordance with one preferred exemplary embodiment of the system 520, the electrical power source 528 is in the form of a battery pack BP composed of two non-rechargeable AAA alkaline batteries B1 and B2 which are connected in series to produce an open circuit voltage of 3.3 VDC. A battery insulator pull tab 532 that comprises a strip of an electrical insulating material is interposed between one of the poles of the battery pack to isolate the batteries B1 and B2 from the elongated light source 526. Thus, the pull tab 532 can be thought of as a single use OFF/ON switch like shown schematically in FIG. 31, which when the tab is in place the switch is in its Off state to prevent the batteries from applying electrical power to the LEDs of the elongated light source 526. Removal of the pull tab by the user of the instrument effectively “closes” the ON/OFF switch to apply the electrical power from the batteries B1 and B2 to the elongated light source 526 via the electrically conductive cable 534. That action energizes the elongated light source. Once energized the elongated light source will produce visible light for at least one hour. The cable carrying the electrical power to the elongated light source is preferably composed of a pair of 30 AWG electrical conductors C1 and C2. In this exemplary embodiment conductor C1, which is connected to the negative terminal of the battery pack, is a tinned copper conductor, whereas conductor C2, which is connected to the positive terminal of the battery pack, is bare copper. It should be noted that in lieu of using the pull tab 532, the lighting subsystem may be constructed to use any type of conventional ON/OFF switch to form the ON/OFF switch shown in FIG. 31.

As best seen in FIGS. 30 and 34-36 the elongated light source 526 basically comprises an elongated strip in the form of a flexible circuit having a longitudinal axis X. The flexible circuit strip is disposed and sealed within the encasement sleeve 538 (FIGS. 30 and 32) to protect the components of the flexible circuit from body fluids when the suction tube is disposed within the patient's vagina. The details of the construction of the flexible circuit 536 will be described later. Suffice it for now to state that flexible circuit includes the heretofore identified plurality of light-emitting diodes D1-D14, resistors R1 and R2, and various electrical conductor segments connecting those components to one another. The LEDs are arrayed sequentially at equidistant spacing on the strip and are configured to emit visible, e.g., white, light. It should be noted that while the light source 526 shows a total of fourteen LEDs the strip can include more or less LEDs depending upon the application. In the exemplary embodiment shown the LEDs are configured in a 300 mm long array with 25 mm spacing between them.

The encasement sleeve 538 is preferably a flexible tube formed of medical grade polyurethane which is clear or otherwise transparent so that the light emitted by the light emitting diodes can freely pass therethrough. The flexible circuit 536 is disposed in the encasement sleeve by introducing the distal end of the flexible circuit into the proximal end of the encasement sleeve, which is open at that time. As best seen in FIG. 35 a hole or window 540 is located immediately adjacent the distal end of the flexible circuit. That hole provides a means for grasping the distal end of the flexible circuit to pull it into the encasement sleeve until its distal end is located adjacent the open distal end of the encasement sleeve. Once in that position the open distal end of the encasement sleeve is permanently closed by squeezing the distal end so that it is flat and heat sealing the abutting surfaces of the flattened distal end of the encasement sleeve together to form a fluid-tight joint 542 (FIG. 27). The conductors C1 and C2 exit from the proximal end of the flexible circuit strip and hence pass out the open proximal end of the encasement sleeve. As shown in FIG. 32 a sealant, such as a UV curable silicone, is introduced into the interior of encasement sleeve from the open proximal end to form a sealed joint 544 through which the conductors C1 and C2 of the cable extend. Accordingly, the flexible circuit is completely enclosed and isolated in the sleeve between the joints 542 and 544 and so that body fluids cannot gain access to the flexible circuit when the suction tube is located within the patient's vagina.

The array of parallel connected LEDS is physically oriented so that the visible light produced from those diodes projects outwardly along the longitudinal axis X and through the sidewall of the encasement sleeve. This feature is illustrated in FIG. 37, wherein the visible light produced by the LEDs D1-D14 are graphically illustrated as cones of light designated by the reference numbers L1-L14, respectively.

Turning now to FIG. 31 the electrical connection of the components making up the illumination system 524 will now be discussed. Thus, as can be seen, one side of the resistor R1 is connected to a solder joint J1, which is connected to the distal end of the cable conductor C1. The opposite side of the resistor R1 is connected to the cathodes of the LEDs D1-D14. One side of the resistor R2 is connected to the solder joint J1. The opposite side of the resistor R2 is connected to the cathodes of the LEDs. The anodes of the LEDs are connected to a solder joint J2. The solder joint J2 is connected to the distal end of the cable conductor C2. The proximal end of the cable conductor C1 is connected to the negative terminal of the battery pack BP by solder joint J3. The proximal end of the cable conductor C2 is connected to the positive terminal of the battery pack by a solder joint J4. As mentioned earlier the battery pack is preferably composed of two alkaline AAA batteries B1 and B2. The resistors R1 and R2 are provided to drop the voltage from the batteries and limit the current provided to the diodes to protect the diodes while enabling them produce visible light of a sufficient intensity to enable users to readily visualize the patient's uterus and adjacent internal anatomy.

A portion of the layout of the components of the flexible circuit 536, e.g., D1-D14, R1, R2 and the electrical conductor sections CS which connect those components, are shown in FIG. 35 and that layout is produced by using a Gerber file layout.

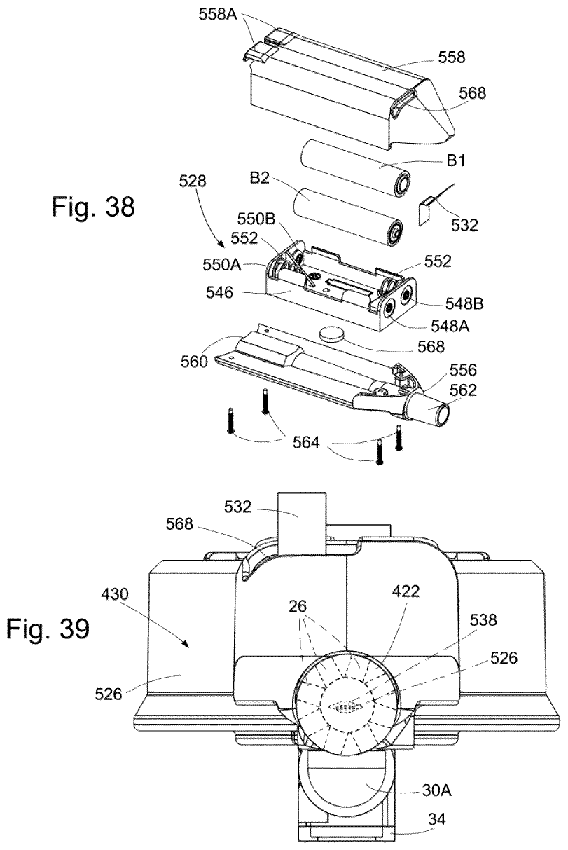

Turning now to FIGS. 38 and 39 the details of the battery pack BP and its associated components will now be described. The batteries B1 and B2 making up the battery pack are connected in series and held in a conventional battery case or receptacle 546, which in turn is mounted and enclosed within a housing to be described later. The battery case includes a pair of electrical contacts 548A and 548B located at the distal end of the case 546 and which are electrically connected. The contact 548A is configured to be engaged by the positive terminal of the battery B1. The contact 548B is configured to be engaged by the negative terminal of the battery B2. The opposite, i.e., proximal, end of the battery case includes another pair of electrical contacts 550A and 550B. A helical compression spring 552 is interposed between the negative pole of the battery B1 and the electrical contact 550A so that the negative pole of that battery is in electrical continuity with that contact. Another helical compression spring 552 is interposed between the positive pole of the battery B2 and the electrical contact 550B so that the positive pole of that battery is in electrical continuity with that contact. The proximal end of the conductor C1 is connected to the electrical contact 550A by solder joint J3. The proximal end of the conductor C2 is connected to the electrical contact 550B by solder joint J4. Thus, the springs 552 bias the batteries B1 and B2 in series and into electric continuity with electrical contacts forming the positive and negative poles of the battery pack.

The battery case with its batteries B1 and B2 is mounted within a housing 554. The housing 554 is composed of a base 556 and a cover 558. The base includes a socket 560 configured for receipt of the port 30A of the suction regulator 430. That action secures the housing 454 to the suction regulator. A passageway (not shown) extends from the socket in the base 556 of the housing to a tubular port 562 at the distal end of the base 556. The tubular port 562 is configured to receive the open proximal end of the suction tube 422 to provide the regulated suction into the proximal end of the suction tube and down its passageway 22A to the array of apertures 26. The cover 558 of the housing 554 is a hollow member which is secured to the base 556 via four nail screws 564 extending through aligned holes in the base and cover. The cover includes a pair of tabs 558A projecting proximally at the top of the cover 558. The tabs are configured to engage contiguous portions of the top surface of the top wall of the suction regulator to assist in the securement of the housing 554 to the suction regulator. When so secured the battery pack is enclosed within the interior of the housing. A disk 566 formed of silicone foam with an adhesive backing is disposed between the bottom surface of the battery case 546 and the base of the housing to act as vibration insulator to prevent the battery case from rattling within the housing 554.

The cover 558 includes a slot 568 through which a portion of the power source actuator or pull tab 532 extends so that the portion of the pull tab extending out of the housing can be readily grasped by a user and pulled through a slot so that its interior end is no longer interposed between the positive (+) pole of battery B1 and the electrical contact 548A. Accordingly, when it is desired to energize the illumination circuitry of the system 520 all that is required is for the user to grasp the exposed end of the pull tab 532 that extends out of the slot 568 to pull it outward, so that electrical power is delivered to the light emitting diodes to cause them to illuminate.

As mentioned earlier the proximal end portion of the conductors C1 and C2 of the cable 434 are connected via solder joints J3 and J4 to the respective negative and positive terminals of the battery pack. As best seen in FIG. 34 portions of the conductors C1 and C2 adjacent the proximal ends of the cable 534 are bent into a somewhat sinusoidal or wavy shape to be able to flex to accommodate the flexing of the flexible circuit 526 and the associated cables as the suction tube is inserted into the patient and positioned for treatment.

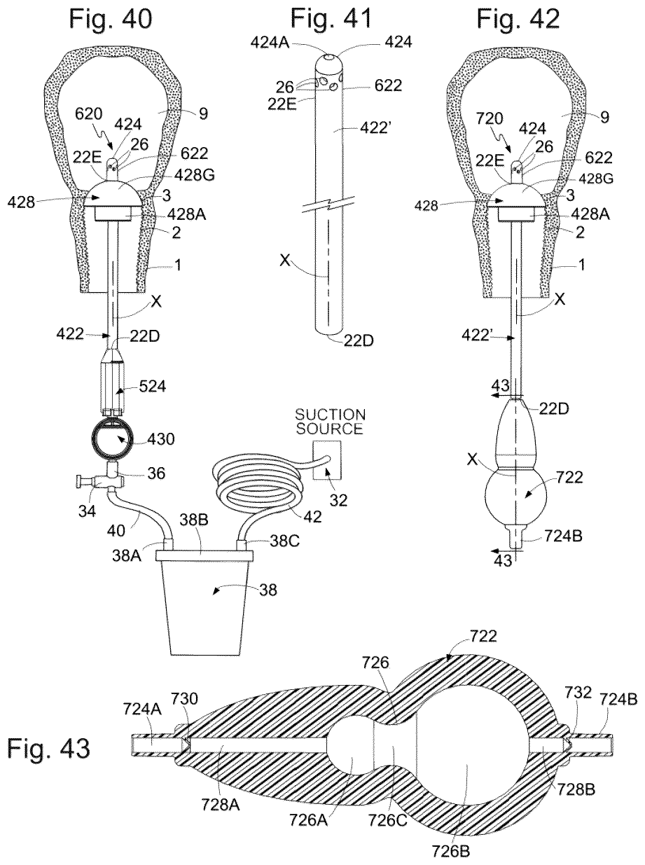

Turning now to FIG. 40, there is shown still another alternative embodiment of a PPH device 620 constructed in accordance with this invention. The PPH device 620 is similar in construction and use as the PPH device 520, except for a change in the construction of the suction tube 422. In particular, the suction tube of the PPH device 620 is in the form of a suction tube 422′ whose distal end portion includes a short nub 622 configured to be located within the patient's uterus proximally of the sealing member 428. In the interest of brevity, the common features of the PPH device 620 with the PPH device 520 will be given the same reference numbers and the details of the construction and the operation of the common features of those devices will not be reiterated. Like the PPH device 520, the PPH device 620 can be used by connecting it to the receptacle or canister 38 and the suction source 32 via the tubing sections 40 and 42 as shown in FIGS. 1A, 1B, and 1C.

As can be seen in FIGS. 40 and 41 the distal end of the suction tube 422′, which is configured for intrauterine placement, is much shorter in length to form a nub 622 which is more easily inserted into the uterus when the cervix is not dilated, as may be the case following cesarean deliveries. In the exemplary embodiment shown the length of the nub 622 is approximately 4 cm measured from the atraumatic tip 424 to the intermediate point 22E of the suction tube 422′. The apex or distal end of the atraumatic tip includes an opening 424A (FIG. 41) which is axially aligned with the central longitudinal axis of the suction tube and serves as the open distal end of the central passageway 22B. Like the suction tube 22 described above, the suction tube 422′ includes a plurality of radially extending apertures 26 located in an array extending through the sidewall 22A of the suction tube from its atraumatic tip to an intermediate point 22E. In this case there are eight radially extending apertures 26, in the form of a first group of four such apertures equidistantly spaced from each other about the circumference or periphery of the nub closely adjacent the apex of nub, and a second group of another four apertures located closely proximately the first group. As in the case of the apertures 26 of the other embodiments of the PPH devices described above, the radius of each of the apertures of each adjacent group is offset by an angle of 45 degrees from the immediately adjacent set. In the sealing device 428 can be pressed against the outside wall of the cervix with the nub 622 extending into the uterus allowing vacuum to enter the uterus to achieve compression. The nub 622 with its reduced number of apertures 26 serves to ensure vacuum is not inadvertently applied within the cervix or vagina when the PPH device 620 is used.

Turning now to FIG. 42 there is shown still another alternative embodiment of a PPH device 720 constructed in accordance with this invention. The PPH device 720 is similar in construction as the PPH device 620, except that it doesn't include the illumination system 524. In addition, the embodiment 720 makes use of a different means for applying suction to the suction tube 422′ and from there to the uterus when the device is used. That different suction applying means is in the form of a manually operable pump 722 in lieu of the suction regulator and the associated components, e.g., on/off selector valve 34, the receptacle 38, the tubing sections 40 and 42 and the suction source 32 of the device 620. In the interest of brevity, the common features of the PPH device 720 with the PPH device 620 will be given the same reference numbers and the details of the construction and the operation of the common features of those devices will not be reiterated.

The manually operable pump 722 is best seen in FIG. 43 and basically comprises a hollow member or bulb formed of any suitable flexible material, e.g., silicone. The bulb is manually compressible so that it can be manually compressed by a user of the PPH device 720 during use of that device. In the exemplary embodiment of the PPH device 720 the bulb is a dual lobed device that includes a distally located lobe section 722A and proximately located lobe section 722B. A central chamber 726 is located in the interior of the bulb and centered on a central longitudinal axis X. The chamber 726 itself is dual-lobed, e.g., it includes a relatively small diameter, e.g., 2 cm, generally spherically shaped chamber section 726A located within the lobed section 722A, and a relatively large diameter, e.g., 6 cm, generally spherically shaped chamber section 726B located within the lobed section 722B. The two chamber sections 726A and 726B are interconnected and in fluid communication with each other via an intermediate section 726C merging the sections 726A and 726B together. The distally located bulb section 722A terminates at cylindrical connector tube section forming an inlet port 724A. The proximally located bulb section 722B terminates at cylindrically shaped connector tube section that forms an outlet port 724B. The connector tube forming the inlet port 724A is configured to be received within the open proximal end 22D of the central passageway 22A of the suction tube 422′. The connector tube forming the outlet port 724B is configured to be received within an open distal end of a section of a conventional flexible tubing (not shown) which is connected to a canister or receptacle (not shown) for receiving and collecting blood and other debris from the uterus of the patient when the PPH device 720 is used. In particular, the connector 724B of the pump 722 can be connected to one end of a conventional tubing section, whose other end is connected an inlet port of any suitable canister or receptacle.

The distal end of the bulb section 722A is in form of a passageway 728A extending distally along the longitudinal axis X from the distally located lobed chamber 726B. A one-way inlet valve 730 is located at the distal end of the passageway 728A and is interposed between that passageway and the inlet port connector 724A. The inlet valve 730 is normally closed and configured when closed to isolate the inlet port 724A from the interior chamber 726 when the pump 722 is in its normal quiescent state, like shown in FIGS. 42 and 43. The proximal end of the bulb section 722B is in form of a passageway 728B extending proximally along the longitudinal axis X from the proximally located lobed chamber 726B. A one-way outlet valve 733 is located at the proximal end of the passageway 728B and is interposed between that passageway and the outlet port connector 724B. The outlet valve 732 is normally closed and configured when closed to isolate the outlet port 724A from the interior chamber 726 when the pump 722 its in its normal quiescent state.

Use of the pump 722 when the PPH device is in place in the patient like shown in FIG. 42 is as follows. The bulb section 722B is manually squeezed by the user. The squeezing of the bulb causes any air that is in the interior chamber portions 726A and 726B to attempt to pass through the passageways 724A and 724B, as shown in FIG. 43, whereupon air within the interior chamber 726 is forced into the passageway 728B to the one-way outlet valve 732, whereupon that valve opens to enable the air to pass out of the pump through the outlet port 724B into the flexible tubing section and from there to the receptacle. However, the closed one-way inlet valve 730 prevents the air from flowing into the inlet port 724A into the suction tube 422′. Thus, the air from within the interior chamber only exits the outlet port 732. Once that has occurred, the user of the PPH device 720 can release the squeezing action on the bulb. The release of pressure on the exterior of the bulb enables the bulb to start to resume its normal quiescent state, whereupon the one-way outlet valve 732 closes, thereby blocking off the outlet port 724B. At the same time, the inlet port 730 opens, thereby producing a partial vacuum or suction in the interior chamber 726B. That suction is coupled through the passageway 728A and the now open one-way inlet valve 730, whereupon the suction passes through the inlet port 724A to the interior passageway 22A of the suction tube 422′ and from there to the apertures 26 in the nub 622 thereby applying the suction to the interior of the patient's uterus. That action will draw any blood and other debris from the uterus through the suction tube 422′ and into the chamber 26 in the pump. After complete release of the squeezing pressure on the pump, the bulb will reassume its normal quiescent state, wherein the inlet and outlet one-way valves are closed.

If a single squeeze and subsequent release of the pump does not suffice to treat the patient, additional suction can be readily accomplished by the user merely re-squeezing the bulb of the pump as described above. That re-squeezing action will cause the one-way outlet valve 732 to open, whereupon blood and debris within the chamber 726 will be forced out of the chambers via the passageway 728B, the now open one-way outlet valve 732, and the outlet port 724B into the tubing section and from therein to the collection receptacle. Once that has occurred the user can then release the squeezing pressure on the bulb 722, whereupon the bulb will assume its normal quiescent state, thereby producing the partial vacuum or suction within the chamber 726 and through the passageway 728A, to cause the one-way inlet valve 730 to open, whereupon the suction will be applied to the patient's uterus via the suction tube 422′, as described above. The squeezing/releasing action can be repeated as many times as necessary to adequately treat the patient, whereupon blood and any other bodily fluid or debris (e.g., placental fragments, small clots, etc.) is withdrawn out of the female patient by the squeezing/releasing action may be transferred into the canister or receptacle for collection and disposal.

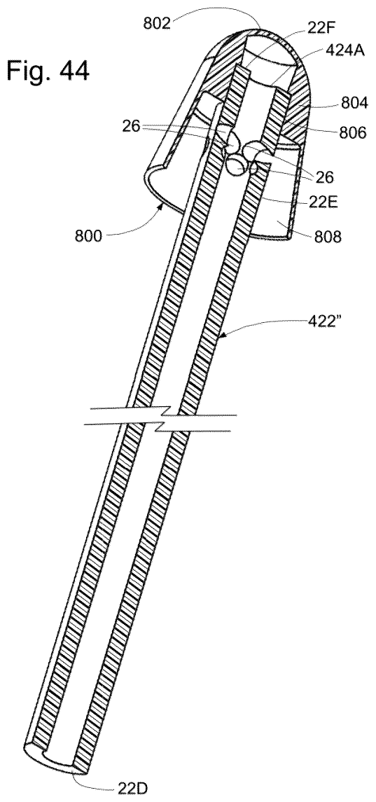

Turning now to FIG. 44 there is shown another alternative embodiment of a suction tube constructed in accordance with this invention. That alternative suction tube is designated by the reference number 422″ and is identical in construction to the suction tube 422′, except for the inclusion of a shield 800 mounted on the distal end of the suction tube 422″. In the interest of brevity, the common features of the suction tube 422″ with the suction tube 422′, will be given the same reference numbers and the details of the construction and operation of the that suction tube will not be reiterated. Thus, as can be seen in FIG. 44, the distal end of the suction tube 422″ includes the heretofore identified shield 800. The shield is a generally conically shaped member having a dome shaped apex 802 forming the distal free end of the shield. The dome shape is provided so that the distal end of the nub is atraumatic. The interior of the dome shaped apex includes a thickened wall portion 804 defining a central bore 806 which is configured to tightly receive and secure the distal end 22F of the suction tube 422″ to the shield. The distal end 22F of the suction tube 422″ is planar and includes a central opening 424A. The nub portion 622 of the suction tube 422″ is located immediately adjacent the distal end 22F of the suction tube and includes the heretofore identified array of the eight apertures 26. The bottom of the thickened wall portion 804 of the shield is in the form of an outwardly flaring skirt 808 whose outer surface merges and is flush with the outer surface of the wall portion 804. The bottom edge of the skirt 808 is located adjacent the intermediate portion 22E of the suction tube 422″. As such, the skirt covers and surrounds the apertures 26 in the suction tube 422″ Thus, the skirt acts as an obstacle preventing or at least deterring debris from gaining access to the apertures 26, which could result in their blockage.

It should be noted that all configurations of the PPH devices of this invention as discussed above, and any variations thereof not specifically described above, are designed and configured to be used in the same step-by-step procedure as described above regardless of whether the PPH device is used for treatment or as a prophylactic therapy. In both treatment and prophylactic use, the PPH device will evacuate blood and compress the uterus. In either uses, if the device doesn't include its own suction source, e.g., a pump, etc., the PPH device may be connected first to a wall canister which then gets connected to the vacuum source. If the PPH device includes it own suction source, e.g., a pump, the pump would be utilized to deliver the desired level of suction to the patient's uterus. The only difference between the two methods of use is the timing. For postpartum hemorrhage treatment, the clinician will defer treatment until there is considerable blood loss, typically 500 ml-1000 ml. For prophylactic use, there is no delay or amount of blood loss necessary to initiate treatment. Moreover, for those PPH devices including the illumination systems like those described above or modifications of such systems, the light could be activated prior to insertion of the PPH device to facilitate placement or inspection of the vaginal canal. The user may also decide to activate the light once the PPH device is in place to illuminate damaged or injured tissue in need repair.

It must be pointed out at this juncture that various changes can be made to PPH devices constructed in accordance with this invention from the exemplary embodiments described specifically above. For example, a generally conically shaped sealing member of this invention may incorporate more tiers to accommodate the range of sizes of female anatomy or may incorporate fewer tiers to facilitate placement. The sealing member may be a single tier with an extended portion (e.g., skirt) adjacent the apex, like shown in FIGS. 9-23 to facilitate closing off the vagina from the ambient atmosphere. The sealing member may be constructed of multiple separate tiers that are assembled on the suction tube. In any case, the overall size and shape of the sealing member may be adjusted to accommodate the wide range of female anatomy and facilitate clinical use of the device. The sealing member may be a solid member, instead of a hollow member. In which case the central passageway of the suction tube will extend through the solid sealing member, with the distal portion of the suction tube projecting outward distally from the apex of the sealing member and with the proximal portion of the suction tube projecting outward proximally from the bottom of the sealing member. If the sealing member is solid, it should still be preferably, but not mandatorily, somewhat flexible in the interest of ensuring that a good seal is made with the patient's anatomy to maintain vacuum in the uterus while being comfortable. Moreover, such flexibility enables the physician to flex the proximal portion of the sealing member to provide access to a portion of the patient's anatomy the physician wishes to address as discussed above and to facilitate removal of the sealing member from the vagina. The suction tube itself may have a single opening at the distal end as opposed to an array of apertures or holes like described above. Further still, the PPH device, itself, may be constructed without an intrauterine tip 24 at the distal end of the tube suction.

Various changes can be made in the methods of managing postpartum hemorrhage or any type of uterine hemorrhage in accordance with this invention. For example, it must be pointed that the three exemplary methods of use as shown and described with reference to FIGS. 1A-1C are merely illustrative of various positions that the sealing member 28, 228, 328 or 428 may be located with respect to the patient's vagina. In particular, the sealing member may be located at any position between the position shown in FIG. 1A and the position shown in FIG. 1C, depending upon the anatomy of the patient and the desires of the medical personnel treating the patient for postpartum hemorrhage. To that end, the size and shape of the sealing member is designed so that it will accommodate a wide range of sizes of female anatomy and minimize the likelihood of unintended insertion into the cervix (a feature that cannot be said of an expandable balloon like that of the Jada® system).

Without further elaboration the foregoing will so fully illustrate our invention that others may, by applying current or future knowledge, adopt the same for use under various conditions of service.

We claim:

1. A device for preventing or treating uterine bleeding or hemorrhage of a female patient having a uterus comprising:
a suction tube or conduit comprising an elongated sidewall having a periphery, a distal free end portion, a proximal end portion, a passageway extending about a longitudinal axis within said sidewall, and at least one aperture in fluid communication with said passageway located at either said distal free end portion of said sidewall or at another portion of said sidewall closely adjacent said distal free end portion, or at said distal free end portion of said sidewall and said another portion of said sidewall;
at least one light emitting component coupled to said suction tube and configured to be illuminated, whereupon light from said light emitting component provides light outside of said suction tube;
a sealing member having a sidewall, an apex at a top of said sidewall and an exterior surface located proximally of said apex, said distal free end portion of said suction tube extending outward distally of said apex;
said device being configured to enable said distal free end portion of said suction tube to be positioned into the uterus of the female patient such that said at least one aperture is confined to the interior of the uterus, said sealing member being configured to be positioned with respect to the uterus so that tissue of the female patient conforms around and engages said exterior surface of said sealing member, said light emitting component illuminating tissue of the female patient, said proximal end of said suction tube being configured so that suction can be applied thereto from a suction source and through said passageway to said at least one aperture to apply suction through said at least one aperture into the uterus of the female patient, whereupon said engagement of said exterior surface of said sealing member with said tissue of the female patient forms a seal therebetween to maintain suction within the uterus.

2. The device of claim 1 wherein said light emitting component is located within said sidewall, and said sidewall is configured to enable light to pass out of said sidewall.

3. The device of claim 1 wherein said at least one aperture comprises an array of apertures extending through said distal free end portion of said sidewall about the entire periphery of said sidewall along a predetermined length of said suction tube at said distal free end portion.

4. The device of claim 1 wherein said sealing member is slidably mounted on said suction tube.

5. The device of claim 3 wherein said predetermined length of said array of apertures is in the range of approximately 4 cm to 14 cm.

6. The device of claim 1 wherein said sidewall of said sealing member is flexible.

7. The device of claim 1 wherein said at least one light emitting component comprises a series of light emitting diodes.

8. The device of claim 7 wherein said series of light emitting diodes is located within said central passageway of said suction tube, and wherein at least a portion of said sidewall of said suction tube is transparent or translucent to enable light from said light emitting diodes to pass therethrough.

9. The device of claim 7, wherein said device additionally comprises a source of electrical power to provide electricity to said series of light emitting diodes, said source of electrical power being located adjacent said proximal end portion.

10. The device of claim 1 additionally comprises a suction regulator coupled to distal end portion of said suction tube to regulate suction provided by the suction source and to provide regulated suction through said passageway to said at least one aperture.

11. The device of claim 1 wherein the suction source comprising a manually operable pump configured to be coupled to said proximal end of said suction tube to apply suction to said suction tube.

12. The device of claim 11, wherein said manually operable pump comprises a hollow squeezable body including an interior chamber having an inlet port and an outlet port, said inlet port being coupled to said proximal end of said suction tube via one-way inlet valve, said outlet port being coupled to the ambient atmosphere via a one-way outlet valve.

13. The device of claim 2, wherein the suction source comprising a manually operable pump configured to be coupled to said proximal end of said suction tube to apply suction to said suction tube.

14. The device of claim 3 additionally comprising a shield located adjacent said apex of said suction tube to deter the blockage of said array of apertures by debris.

15. The device of claim 14 wherein said shield comprises a generally conically shaped member having a peripheral skirt portion overlying said array of apertures.

16. A method for preventing or treating uterine bleeding or hemorrhage in a female patient, the female patient having a uterus having an interior and an opening, said method comprising:
providing a suction tube comprising an elongated sidewall having a periphery, a distal free end portion, a proximal end portion, a passageway extending about a central longitudinal axis within said sidewall, at least one aperture in fluid communication with said passageway located at either said distal free end portion of said sidewall or a portion of said sidewall closely adjacent said distal free end portion, said suction tube including at least one light emitting component coupled to said suction tube and configured to be illuminated, whereupon light from said light emitting component provides light outside of said suction tube;
providing a sealing member having a sidewall, an apex at a top of said sidewall and an exterior surface located proximately of said apex;
inserting said distal end portion of said suction tube into the uterus of the female patient such that said at least one aperture is confined to the interior of the uterus;
positioning said sealing member with respect to the uterus so that tissue of the female patient at the opening of the uterus conforms around and engages said exterior surface of said sealing member;
causing said light emitting component to illuminate tissue of the female patient;
applying suction to said proximal end of said suction tube to apply suction through said at least one aperture into the uterus of the female patient, whereupon said engagement of said exterior surface of said sealing member with said tissue of the female patient outside the uterus forms a seal therebetween to maintain suction within the uterus.

17. The method of claim 16 wherein said light emitting component is located within said sidewall, and said sidewall is configured to enable light to pass out of said sidewall.

18. The method of claim 16 additionally comprising: providing a receptacle or canister for collection of fluid or other body materials resulting from the application of suction to the uterus of the patient.

19. The method of claim 16 wherein said suction is applied to the uterus continuously or intermittently.

20. The method of claim 16 wherein said suction is applied to the uterus by a manually operable pump.
